(19) United States
(12) Patent Application Publication
(10) Pub. No.: US 2016/0010152 A1
(43) Pub. Date: Jan. 14, 2016

(54) Dual Probe:Antiprobe Compositions for DNA and RNA Detection

(71) Applicants: David A. Shafer, Atlanta, GA (US); James L. Murray, Monroe, GA (US); Peixu Hu, Atlanta, GA (US)

(72) Inventors: David A. Shafer, Atlanta, GA (US); James L. Murray, Monroe, GA (US); Peixu Hu, Atlanta, GA (US)

(73) Assignee: GENETAG TECHNOLOGY, INC., Atlanta, GA (US)

(21) Appl. No.: 14/866,185

(22) Filed: Sep. 25, 2015

Related U.S. Application Data
(63) Continuation of application No. PCT/US2014/031898, filed on Mar. 26, 2014.
(60) Provisional application No. 61/805,272, filed on Mar. 26, 2013.

(51) Int. Cl. C12Q 1/68

(52) U.S. Cl. CPC C12Q 1/6876; C12Q 2600/16

(57) ABSTRACT

Provided herein are multi-probe systems and methods for amplifying and/or detecting multiple nucleic acid targets in a sample. The multi-probe systems comprise two or more probes having identical and/or different labels and are used together to target two or more related signature sequences in a genus, a species, or a gene. The probes comprise one or more probe:antiprobe systems selected from iDDS probes, iDDS-2Q probes, MacMan probes, Flip probes, Universal probes, Half-Universal probes, ZIPR probes, and G-Force probes. The probes are generally flanked by one set of primers, one primer, one primer-probe, or two primers. The methods utilize the multi-probe systems in real time PCR analysis for facilitating the assessment and/or diagnosis of nucleic acid sequences by providing confirmation of target detection and/or by providing greater signaling per test.

### CROSS-REFERENCE TO RELATED APPLICATIONS

This application is a continuation of international application PCT/US2014/031898, filed Mar. 26, 2014, which claims benefit of priority under 37 C.F.R. §1.119(e) of provisional application U.S. Ser. No. 61/805,272, filed Mar. 26, 2013, now abandoned, the entirety of both of which are hereby incorporated by reference.

### BACKGROUND OF THE INVENTION

1. Field of the Disclosure

This disclosure relates to the field of nucleic acid probe technology, and more specifically to compositions and methods to identify and quantify targeted DNA or RNA sequences. In particular it relates to the labeling and detection of normal or mutant sequence targets of scientific or clinical interest, either during or following amplification processes.

2. Description of the Related Art

The detection of targeted polynucleotide sequences is commonly based on methods that hybridize labeled DNA probes to a target sequence of interest. To work effectively, the probe-target hybridization products must be washed after hybridization to remove unbound probes and probes that are weakly bound to non-specific targets. However, under the conditions of real-time PCR (U.S. Pat. Nos. 4,965,188, 5,210,015, 5,487,972, and 5,538,848), a wash step is not feasible, and thus novel probes had to be devised that selectively generate signaling when they are bound to a matching target and that have diminished or quenched signaling when they are unbound and floating free in solution. To achieve this end, there has been reliance on probes that employ the excitation and transfer of fluorescent energy between a donor and an acceptor molecule, such as between two fluorophores, or between a fluorophore and a quencher (Didenko V, 2001, Biotechniques 31:1106-1116, 1118, 1120-1121; Chen et al., 1997, Proc. Natl. Acad. Sci. USA 30: 94: 10756-10762). The fluorescence emission spectrum of the donor overlaps the absorption or excitation spectrum of the acceptor, and the excited-state energy of the fluorescent donor molecule is transferred to the acceptor molecule when in close proximity (10 to 100 angstroms). When the acceptor molecule is fluorescent, it provides an emitted signal at a longer wavelength. If the acceptor molecule is a quencher, fluorescent signaling is significantly diminished and may be essentially turned off.

TAQMAN® and molecular beacon probes are common probes of this type for real-time PCR detection. In both cases, they serve as an internal probe used in conjunction with a pair of primers that flank the target region. The primers amplify the target segment and the probe selectively binds to an identifying sequence in between, thereby causing increases in fluorescent signaling relative to target frequency. While these probe systems are similar in effect, they employ different detection mechanisms.

A TAQMAN® probe comprises a short oligonucleotide that complements a target sequence, and which is labeled on opposing ends with a fluorescent donor and an acceptor (U.S. Pat. No. 5,538,848). Typically, a fluorophore such as fluorescein is paired with a longer wavelength fluorophore (e.g. TAMRA®) or a non-fluorescent quencher such as a BLACK HOLE QUENCHER®. While the TAQMAN® patent has essentially expired, this technology still remains the dominant probe system for real time PCR.

Molecular Beacon probes use similar fluorescent interactions to detect PCR products, (U.S. Pat. No. 5,925,517; Tyagi et al., 1996, Nat. Biotechnology 14: 303-308), but they include short complementary ends that bind together, forming a stem-loop structure where the quencher and fluorophore-labeled ends are brought closely together and signaling is highly suppressed.

SCORPION® probes also provide a stem-loop detection mechanism, except that the probe also has a spacer and a primer attached (Whitcombe et al., 1999, Nat. Biotechnol. 17: 804-807; U.S. Pat. No. 6,326,145). The probe maintains a quenched stem-loop configuration in the unhybridized state, but when denaturation occurs again followed by annealing, the probe region binds to the template, opening the stem-loop structure, and fluorescence is released.

Similar to SCORPION®, SUNRISE® probes comprise a primer attached to a hairpin probe that is extended during amplification. This separates the internal quencher label from the 5′ terminal fluorophore (Nazarenko et al., 1997, Nucl. Acids Res. 25: 2516-2521). Other probes for real-time PCR include: (a) Dual Hybridization probes (Wittwer et al., 1997, BioTechniques 22: 130-138) involving two fluorescent probes binding to the target side by side and based on fluorescent energy transfer between the two adjacent labels; and (b) LUX primer-probes (Kusser, 2006, Methods Mol. Biol. 335: 115-133) involving a 3′ fluorophore-labeled primer that self-quenches by folding and binding to a short complementary sequence that has been appended to the 5′ end of the primer sequence.

Conventional dual-labeled probes require selective design and are costly. Their synthesis is difficult and they require manual post-synthesis addition of at least one label as well as high pressure liquid chromatography purification. TAQMAN® and molecular beacon probes also require two opposing primers that flank the probe. To function effectively during the annealing step, TAQMAN® and molecular beacon probes must be longer and have a Tm (melting temperature) that is 5 to 10 degrees higher than the primers since the probe must bind firmly to the target before extension. This requirement makes it difficult to design or develop dual-labeled probes that can selectively detect SNPs (single nucleotide polymorphisms) or single base mutations, and consequently, false positives are a common problem.

FISH (fluorescent in situ hybridization) probe techniques require: 1) labeled DNA probes, 2) probe hybridization to fixed, denatured targets, 3) the stringent washing of unbound probes, and 4) fluorescent excitation and detection (Barch M. J, editor. The ACT Cytogenetics Laboratory Manual 2nd ed. New York: Raven Press; 1991).

Microarray detection resembles FISH detection. Arrays are typically based on printing substrates with bound oligonucleotide cDNA probes, applying fluorescent-labeled DNA or RNA targets that hybridize to the probes, washing stringently, and detection, usually by laser scanning (Schena et al., (1995) Science 270: 467-470; Heller et al., (1997) Proc. Natl. Acad. Sci. U.S.A. 94: 2150-2155). Like FISH, the wash steps are complex and time consuming. More importantly, preparing and labeling targets is costly since each target sample is unique, and therefore, microarray-based assays have had limited value for clinical diagnostics.

There is a need in the art for more effective and reliable probe systems for nucleic acid detection, particularly for qPCR assays employed for molecular diagnostics since such assays are prone to false positives and negatives that can result in harmful treatment decisions.

Particularly, the prior art is deficient in multi-probe systems that can overcome these deficiencies by detecting two or more related diagnostic target sequences in the same assay. The present invention fulfills this longstanding need and desire in the art.

### SUMMARY OF THE INVENTION

The present invention provides probe systems suitable for real-time and end-point detection of DNA or RNA sequences, including probes that can discriminate single base variants such as SNPs and point mutations. The disclosure primarily includes probe systems that are effective for quantitative real-time PCR (qPCR), a process where small gene segments are exponentially amplified and their frequency is detected by fluorescent signaling during amplification. Also provided are multi-probe methods in which two or more such probes are combined together in a single assay, targeting two or more signature sequences in the same genus, species, or gene, in order to improve the certainty and/or the signaling capacity of the test. This technology is of particular value when the primer and probe configuration serves to detect two or more target sequences within the same amplicon. In addition, the invention provides diagnostic compositions of this multi-probe system that serve to identify specific diseases or cancers with greater sensitivity and/or reliability due to the advantage of single assay detection of closely related target sequences. These probe systems can be applied to many different kinds of nucleic acid targets in research, biomedicine, and the life sciences in general.

More particularly, the present invention is directed to a multi-probe system to improve signaling and/or reliability in detection of a genus, a species, a gene or other related nucleic acid targets during real time PCR or other amplification methods. The multi-probe system comprises (i) two or more probes complementary to two or more related signature sequences in a sample, where the probes have identical labels as signal amplifiers, have different labels for confirmation of detection, or have both identical and different labels; wherein the probes comprise one or more probe:antiprobe systems selected from the group consisting of iDDS probes, iDDS-2Q probes, MacMan probes, Flip probes, Universal probes, Half-Universal probes, ZIPR probes, and G-Force probes. The multi-probe system also comprises (ii) two flanking primer components comprising two primers, one primer or one primer-probe, or two primer-probes.

The present invention is directed to a related multi-probe system further comprising one or more alternate probes selected from the group consisting of TaqMan probes, Molecular Beacon probes, Scorpion probes, LUX probes, Sunrise probes and Dual Hybridization probes. Alternatively, one or more probes comprising the multi-probe system are replaced with an alternate probe.

The present invention is also directed towards a method for improving signaling and/or reliability in detecting a genus, a species, a gene, or other related nucleic acid targets. The method comprises obtaining a biological sample from a human, an animal or an organism;

contacting the biological sample with the labeled probes and flanking primer components comprising the multi-probe system described herein; and detecting a signal emitted by the label upon contacting the target in the biological sample. If the labeled probes comprise an identical label, the signal is amplified, thereby improving signaling in detecting the target, or if the labeled probes each comprise a different label, detection is confirmed, thereby improving reliability in detecting the target or a combination thereof.

### BRIEF DESCRIPTION OF THE DRAWINGS

FIG. 1 schematically illustrates the multi-probe system described in example 1 wherein two iDDS-2Q probe: antiprobe systems are used together, targeting two signature sequences that identify the vanA gene of Enterococcus faecium. The first probe (P1-2Q) is directed at an antisense sequence and the other (P2-2Q) is directed at a sense sequence. The black-filled circles indicate quencher molecules that absorb fluorescence, and the white and gray filled circles indicate fluorescent emitting molecules of a different color. In this composition, the two sequence regions targeted by the double probes are encompassed by one set of flanking primers. At typical hybridization conditions during the annealing step of qPCR, these double quencher iDDS probes (iDDS-2Q) preferentially bind to fully matching target templates, thereby releasing fluorescent signaling. The quencher labeled antiprobes (AP1 and AP2) serve to block probe binding to incorrectly matching templates, and in doing so, they turn off probes not bound to a correct target. In addition, the second quencher on the 3′ end of the probes also diminishes the signaling of any unbound probes, so that unwanted background signaling is reduced further. These probes thus provide highly sensitive fluorescent signaling and instant confirmation of target assessment by virtue of simultaneously detecting two related target sequences in parallel.

FIG. 2 shows fluorescent signaling curves generated by qPCR using two iDDS-2Q probes specific to the vanA gene of Enterococcus faecium that confers vancomycin resistance (see the scheme in FIG. 1). The curves are from two tubes wherein each tube contained both vanA probes. The first tube had a vanA positive template, and curves 1 and 2 show amplification and positive signaling from that sample. The second tube had a vanB control template, and the two flat curves shown indicate the absence of vanB amplification and detection. Note that the vanA probe 1 is labeled with an orange fluorophore (CalFluorOrange560) and emits a higher signal than the vanA probe 2 labeled with a red fluorophore (CalFluorRed610). Nonetheless, both positive signals generally start ascent at the same time point in qPCR cycling, and thus, they have about the same CT values. With most qPCR instruments, divergent signaling levels from different fluorophores can be brought into balance by adjusting their gain settings. With this gain adjustment, an extensive validation study of this two-probe vanA assay was conducted with 182 hospital patient samples screened for vanA infection. It was found that 24 positive samples were correctly detected and identified with both iDDS-2Q probes and that the positive signals for both iDDS-2Q probes, as measured by CT values, were found to be essentially identical. This conclusion was evidenced by the instrument-detected CT values, which were very closely correlated (mean difference of ˜0.32 CT, most variant pair ˜0.6 CT).

FIG. 3 shows another example of fluorescent curves generated by qPCR with two iDDS-2Q probes together in one tube (similar to the scheme of FIG. 1); wherein both probe sites were amplified by one pair of primers. Again, the two probes are labeled with different colors and they target two nearby signature sequences in a repetitive DNA segment of Trichomonas vaginalis (T. vag.) that is diagnostic of that infectious disease. Curves 1 and 2 reflect two positive probe signals from one tube containing a T. vag.-positive sample, while the two flat curves are from a second tube containing a human genomic DNA control sample. As with the vanA assay, the positive curves differ in height, but have essentially the same CT values. In another extensive patient validation study with this assay (n=186), 27 patient samples were found T. vag. positive with both probes and their CT values only differed by 0.3 CT (max variance 0.6). This dual iDDS-2Q assay proved three times more sensitive than standard microscope “wet-prep” analysis, which only detected 9 positives.

FIG. 4A shows fluorescent curves generated by qPCR using two iDDS-2Q probes together that are specific for: (a) the EGFR L858R mutation site in exon 21 or (b) the WT EGFR sequence at the L858 site, wherein each probe is labeled differently. Curve 1 reflects detection with the mutant specific probe in a sample containing 100% mutant L858R target sequence. The WT probe (curve 2) did not give a false positive signal although both probes use the same primer set. Thus, this dual probe assay provides clear discrimination of a single base difference.

FIG. 4B shows fluorescent curves generated by qPCR using the same two probes as in FIG. 4A, but run on a different instrument. In this case, the positive sample contained a 50/50 mixture of WT and L858R mutant EGFR templates that were run in one tube with both probes present. The positive curves 1 and 2 are quite similar in angle and height, while the two flat curves reflect the same two probes in a second tube where the sample was a water control.

FIG. 5A shows fluorescent curves generated by qPCR using two iDDS-2Q probes specific for WT human EGFR. Probe 1 detects a variable deletion site in exon 19 commonly associated with lung cancer, and Probe 2 detects an EGFR reference sequence just upstream of the exon 19 deletion site. Each probe is labeled differently, and they are flanked by one primer set. This is an inverse method to detect the mutation frequency, since a regular probe cannot be used to detect a variable deletion target. Probe 1 only detects if the WT sequence is present at the exon 19 target site. It fails to detect when any exon 19 deletion mutation occurs. Known cancer-related exon 19 mutations typically involve an in-frame 15-base deletion, but they can range from 9 to 24 bases. Probe 2, the reference probe, detects all EGFR amplicons present, with or without an exon 19 deletion. Thus, the proportion of Probe 1 signaling to Probe 2 signaling reflects the relative quantity of exon 19 deletion mutants in the sample of interest. In this example, the two-probe reaction contained a 100% WT EGFR target template, and thus both curves ran parallel to one another.

FIG. 5B shows two fluorescent curves generated by qPCR using the same two probes as in FIG. 5A. In this case, the reaction contained a 50/50 mix of both WT and exon 19 deletion mutant templates. Thus, the WT curve runs about 50% lower than the reference probe curve. This assay can easily distinguish patient samples containing 20-50% EGFR mutant cells.

FIG. 5C shows two fluorescent curves generated by qPCR using the same two probes as in FIG. 5A. In this case, the reaction contained a 100% EGFR deletion mutant template. The reference probe, Probe 1, positively detects the EGFR deletion mutant template, despite the mutant sequence being present. In contrast, Probe 2 shows a flat curve indicating that all the amplicons were mutant and none contained the wild sequence at the diagnostic deletion site.

FIG. 6 shows two fluorescent curves generated by qPCR using two iDDS-2Q probes together that are specific for two diagnostic targets in the 5′ untranslated region (UTR) of enterovirus, e.g., echovirus (EV). The probes are flanked by one primer set, and they partially overlap one another in target sequences—one detecting on the sense side and one detecting on the antisense side. Positive curves 1 and 2 reflect the two probes working together in one tube containing an EV positive sample. The two flat curves reflect the two EV probes together in another tube containing a human genomic DNA control sample.

FIG. 7 shows two fluorescent curves generated by qPCR using two iDDS-2Q probes specific for two diagnostic targets in the 5′ UTR of parechovirus (PV). Curves 1 and 2 reflect two positive probe signals in one tube using a PV positive sample. The two flat curves reflect the two PV probes together in another tube that contained a human genomic DNA control sample.

Exemplary embodiments of the methods and systems of the present disclosure are set forth below. Other features, objects, and advantages of the disclosure will be apparent to one of skill in the art upon examining the descriptions, drawings, examples and claims provided. It is intended that all such additional systems, methods, features, and advantages be included within this description, be within the scope of the present disclosure, and be protected by the accompanying claims.

### DETAILED DESCRIPTION OF THE INVENTION

All publications, patents and patent applications cited in this specification are herein incorporated by reference. It is to be understood that this disclosure is not limited to the particular embodiments described, nor to the methods and materials employed, and may vary since the scope of the disclosure is only limited by the claims. Each embodiment described herein has discrete components and features, which may be separated from or combined with the features of any other embodiment without departing from the scope or spirit of the present disclosure. Any recited method can be carried out in the order of events recited or in any other order that is logically possible. Unless otherwise defined, all terms used herein have the same meaning as commonly understood by one of ordinary skill in the art of molecular biology.

The following abbreviations are used: iDDS, internal DNA Detection Switch; qPCR, quantitative PCR (real-time PCR); LNA, locked nucleic acid; BNA, bridged nucleic acid; Tm, melting temperature; SNP, single nucleotide polymorphism; and CT, cycle threshold.

The terms “a” or “an”, when used in conjunction with the term “comprising” in the claims and/or the specification, may refer to “one”, but it is also consistent with the meaning of “one or more”, “at least one”, and “one or more than one”. Some embodiments of the invention may consist of or consist essentially of one or more elements, components, method steps, and/or methods of the invention. It is contemplated that any method, compound, composition, system, or device described herein can be implemented with respect to any other method, compound, composition, system, or device described herein.

The term “or” in the claims refers to “and/or” unless explicitly indicated to refer to alternatives only or the alternatives are mutually exclusive, although the disclosure supports a definition that refers to only alternatives and “and/or”.

The term “about” refers to a numeric value, including, for example, whole numbers, fractions, and percentages, whether or not explicitly indicated. The term “about” generally refers to a range of numerical values, e.g., +/−5-10% of the recited value, that one of ordinary skill in the art would consider equivalent to the recited value, e.g., having the same function or result. In some instances, the term “about” may include numerical values that are rounded to the nearest significant figure.

The terms “DNA amplification” and “amplification” refer to any process that increases the copies of a specific DNA sequence by enzymatic amplification. A commonly used process is the polymerase chain reaction (PCR) as described in U.S. Pat. Nos. 4,683,195 and 4,683,202.

The terms “polymerase chain reaction” and “PCR” as used herein refers to a thermocyclic, polymerase-mediated, DNA amplification reaction employing nucleic acid templates, oligonucleotide primers complementary to the template, a thermostable DNA polymerase, and deoxyribonucleotides.

The term “qPCR” refers to a real-time polymerase chain reaction in which primers and labeled probes are used during the PCR amplification process to simultaneously detect the quantity of a targeted DNA molecule as it is being produced at each step in the reaction.

The term “primer” as used herein refers to an oligonucleotide complementary to a DNA or RNA template to be amplified or replicated. A primer hybridizes or anneals to a template sequence and is used by a polymerase to start the replication/amplification process.

The term “probe” as used herein refers to an oligonucleotide of variable length that is used to detect an identical, similar, or complementary nucleic acid sequence in a targeted template by hybridization. The oligonucleotide probe is typically labeled with a detectable moiety such as radioactive, fluorescent or chemiluminescent compounds.

The term “fluorophore” as used herein refers to any reporter group whose presence can be detected by its light-emitting properties.

The term “quencher” as used herein refers to molecules that interfere with or absorb the fluorescence emitted by a nearby fluorophore.

The term “complementary” refers to the existence of a sufficient number of matching bases between two sequence segments so that they can specifically bind or hybridize together.

The term “denaturation” refers to the separation of complementary DNA strands that form a duplex, typically by heat or denaturant treatment.

The term “hybridization” as used herein refers to the process of association of two nucleic acid strands to form an anti-parallel duplex stabilized by hydrogen bonding between opposing strands. The hybridized strands are called a “duplex.”

The term “hybridization affinity” refers to the degree of chemical attraction between two nucleic acid segments based on the binding of matching base pairs between them. Hybridization affinity varies with the length and sequence of the duplex.

The term “mismatched base” as used herein refers to a duplex in which one or more opposing nucleotide bases do not pair in a complementary manner. A mismatch can be due to addition, deletion, or substitution of a natural or non-natural base, or a spacer.

The terms “target” and “target nucleotide sequence” as used herein refers to a polynucleotide sequence that it is desired to detect.

The term “signature sequence” as used herein refers to a target nucleotide sequence that serves to identify a gene, a species, or an organism of interest.

The term “probe: antiprobe” as used herein refers to a pair of oligonucleotides having nearly or exactly the same number of base positions and having sequences substantially complementary such that, in the absence of a third nucleotide sequence hybridizing to the probe or the antiprobe, said oligonucleotides can form a duplex.

The term “system” as used herein refers to a combination of at least two oligonucleotide components that cooperate together to selectively hybridize to a target nucleotide sequence and to generate a detectable signal. The system may further include primers for the polymerase amplification of a targeted nucleotide sequence.

Generally, the multi-probe systems typically comprise two labeled oligonucleotides, a probe and an antiprobe, that can interact together. In the most general configuration, the probe sequence is complementary to the intended target sequence, and the antiprobe sequence is complementary to the probe except for comprising at least one mismatched base in a non-terminal position. The antiprobe is designed or configured to provide an error checking mechanism for the probe. In most embodiments, the probe is generally labeled with a fluorescent emitter and the antiprobe is generally labeled with a fluorescent modulator, such as a quencher, although such labeling can be reversed and other components, such as a second fluorophore, can serve as a fluorescent modulator. In such embodiments, when probe and antiprobe are bound together, the interacting label moieties are proximate and signaling is diminished, but when the probe binds to a complementary target, fluorophore signaling is released. The most common configuration of this system for qPCR is an iDDS probe, wherein the target-specific probe is labeled with a fluorophore, the antiprobe is labeled with a quencher, and the probe binds to an internal target sequence between a pair of flanking primers. The 3′ end of the probe is blocked to prevent extension. The primers amplify the target segment and the probe detects the amplicons generated, but with high specificity for a particular target sequence. In a key variant, iDDS-2Q probes, the fluorophore-labeled probe component is also labeled with a quencher to further diminish background signaling.

These iDDS probe systems can be configured to enable discrimination of two target sequences that differ by only one base. Accordingly, the probe and antiprobe sequences are engineered to achieve three separate hybridization affinity levels in solution: (i) a first high-affinity level between the probe and the intended target, (ii) a second intermediate-affinity level between the probe and the antiprobe that is determined by the type and position of the mismatch placed in the antiprobe, and (iii) a third low-affinity level between the probe and an incorrect target that differs by at least one base from the sequence of the intended target. The expected hybridization affinity levels are assessed by calculating the Tm and the ΔG of the duplexes expected.

Another probe:antiprobe system is the ZIPR probe system. In a ZIPR probe, the 3′ end of the probe component is not blocked and it serves as a primer-probe where it replaces one primer in an amplification reaction. When a primer-probe is incorporated into an amplicon, antiprobe binding is prevented and fluorescent signaling results. Yet another probe:antiprobe system is the Flip probe system. In a Flip probe system, the antiprobe component is structurally joined to one primer, and this change creates linear versus sigmoid amplification curves by real-time PCR. In yet another probe:antiprobe system, termed the MacMan probe, the probe is 5′ fluorophore labeled and the 3′ end comprises a tail sequence not complementary to the target sequence, and the quencher labeled antiprobe is complementary to the tail sequence of the probe. In yet another probe:antiprobe system, termed the Universal probe system, the probe and antiprobe are generic and comprise sequences not complementary to the target sequence. The target template is first extended with a linker-primer, wherein the linker sequence matches the 3′ end of the Universal probe. The probe then serves as a generic primer-probe that can amplify and label any amplicon extended with the linker-primer. In a related system, termed the Half-Universal probe system, the probe component has a generic fluorophore-labeled 5′ end and a target complementary 3′ primer end. The quencher-labeled antiprobe component is complementary to the 5′ generic sequence of the probe. In yet another probe:antiprobe system, termed the G-Force probe system, one oligonucleotide comprises sequentially: a 5′ fluorophore-labeled C-rich segment, a spacer, a fluorescence-absorbing G-rich segment that serves as an antiprobe, and a 3′ primer segment. When floating, the C-rich and G-rich segments fold and bind together, diminishing signaling. However, when the probe serves as a primer, it is incorporated into an amplicon, it thereby separates the C-rich and G-rich segments, wherein fluorescent signaling is released.

The length, sequence and mismatch placement for the components are designed and configured: (1) wherein the hybridization affinity of the probe to the intended target is higher than the affinity of the probe to the antiprobe, as measured by a difference of about 4 or more degrees C. in Tm and about 2 or more kcal/mol in ΔG; and (2) wherein the affinity of the probe to the antiprobe is higher than the affinity of the probe to the incorrect target (by about another 4 or more degrees C. in Tm and about another 2 or more kcal/mol in ΔG). However, in cases where the inherent thermodynamic difference between the probe/intended target duplex and the probe/incorrect target duplex is limited, the probe may also be modified with an intentional mismatch, advantageously placed about two bases away from the single base variant expected. This probe modification diminishes the hybridization affinity between the probe and the incorrect target due to the proximity of the probe mismatch to the sequence mismatch (the SNP or single base mutant of interest) in the incorrect target. With these various thermodynamic designs, a probe:antiprobe system can achieve discrimination of single base variants and can maintain such discrimination over a wide range of hybridization conditions and annealing temperatures—particularly when employed for real-time PCR.

The compositions and probe systems of the present disclosure provide sensitive and specific detection of DNA or RNA target sequences, particularly for assessing PCR products that are amplified and detected by quantitative real-time PCR (qPCR). The primary embodiment of the disclosure encompasses a multi-probe system for qPCR, or other amplification methods, to improve signaling and or reliability when detecting a genus, a species, a gene or other related nucleic acid targets. The primary system comprises two or more probes working together that are complementary to two or more related signature sequences in a sample, and the system comprises two flanking primer components that include two primers, one primer and one primer-probe, or two primer-probes. With this system, the multiple probes may comprise the same labeling, wherein signaling may be amplified and false negatives may be avoided.

Alternatively, the multiple probes may comprise different labeling, wherein detection of related targets will be confirmed and false positives will be avoided. With this primary multi-probe system, the probe technology employed for one or more probes is selected from the group consisting of: iDDS probes, iDDS-2Q probes, MacMan probes, Flip probes, Universal probes, Half-Universal probes, ZIPR probes, and G-Force probes. All of these probe systems were previously disclosed by the Applicant in at least U.S. Pat. No. 8,076,067 entitled Probe:Antiprobe Compositions and Methods for DNA or RNA Detection; US Publication No. 20120058474, entitled Probe:Antiprobe Compositions and Methods for DNA or RNA Detection; European Patent Application No. 07836913.9 and International App. No. PCT/US2007018167 entitled Probe:Antiprobe Compositions and Methods for DNA or RNA Detection; provisional application U.S. Ser. No. 61/534,925 entitled Probe:Antiprobe Compositions for High Specificity DNA or RNA Detection; International App. No. PCT/US2012/55077 entitled Probe:Antiprobe Compositions for High Specificity DNA or RNA Detection; and non-provisional application U.S. Ser. No. 14/216,413 entitled Probe: Antiprobe Compositions for High Specificity DNA or RNA Detection and are hereby incorporated by reference.

The embodiments of the present invention describe multi-probe systems for qPCR based on iDDS probes, ZIPR probes and Half-Universal probes. In brief, an iDDS probe:antiprobe system typically comprises a fluorophore-labeled probe complementary to the intended target, and a quencher labeled antiprobe that is complementary to the probe except for an intentionally mismatched base in a non-terminal position. The 3′ end of the probe is blocked to prevent extension, and the antiprobe provides both a signaling function and a high specificity, error-checking mechanism. A pair of flanking primers serve to amplify the sequence region containing the target specific probe sequence. During the annealing step of PCR, the probe will preferentially bind to a correct target and it will light up, or it will bind to an antiprobe, whereupon it will be turned off. In either case, this system avoids binding and detecting a closely related but incorrect sequence target. An iDDS-2Q probe is a variant iDDS probe wherein the probe is also labeled with a quencher. This change reduces background and enhances signaling.

ZIPR probes and Half-Universal probes are also based on a probe:antiprobe structure, but they are only suitable for amplification based detection such as real time PCR. A ZIPR probe resembles an iDDS probe and antiprobe in structure, except that the 3′ end of the probe is not blocked and it serves as a primer. When a ZIPR probe is incorporated into an amplicon, fluorescent signaling is released. Half-Universal probes are also primer-probes, and they are based on a Universal probe that consists of a generic probe and antiprobe and a linker-primer which extends the target amplicon with a generic sequence that is suitable for binding the Universal probe. A Half-Universal probe is similar in that it employs a generic sequence to bind the labeled probe and antiprobe components together. However, the 3′ end of the probe is extended with a target specific primer sequence. Again the primer-probe is incorporated into the amplicon and fluorescent signaling is released. Half-Universal probes have an economic advantage over ZIPR probes since they use a generic antiprobe.

In one embodiment of the present invention, a multi-probe system to improve signaling and/or reliability in detection of a genus, a species, a gene or other related nucleic acid targets during real-time PCR or other amplification methods, where the system comprises (i) two or more probes complementary to two or more related signature sequences in a sample, said probes having identical labels as signal amplifiers or having different labels for confirmation of detection or having both identical and different labels; wherein the probes comprise one or more probe:antiprobe systems selected from the group consisting of: iDDS probes, iDDS-2Q probes, MacMan probes, Flip probes, Universal probes, Half-Universal probes, ZIPR probes, and G-Force probes; and (ii) two flanking primer components comprising two primers, one primer or one primer-probe, or two primer-probes.

In this embodiment, the system may further comprise one or more alternate probes selected from the group consisting of TaqMan probes, Molecular Beacon probes, Scorpion probes, LUX probes, Sunrise probes and Dual Hybridization probes; or wherein one probe comprising the system is replaced with the alternate probe. While these probe systems are in routine use and can be multiplexed, i.e., two separate assays in one tube, they have not been employed with two or more probes that target two or more related sequences within a single amplicon.

In this embodiment, the probes or antiprobes further may comprise one or more synthetic nucleotides. For example, the synthetic nucleotide is LNA or BNA nucleotides. In addition, one or more probes may be configured for sequential or separate use in target detection.

In this embodiment, the multi-probe system is suitable for real-time PCR or other amplification methods, to improve signaling and or reliability when detecting a genus, a species, a gene or other related nucleic acid targets. The related target sequences that are detected can be on the same or opposite strands of the same target amplicon, on different amplicons, or on different segments of a targeted gene or species. While greater signaling is always of value, the primary value of this technology is in its ability to separately detect two or more closely related “signature” sequences that confirm the presence of the targeted gene or species. This capability is particularly valuable for clinical diagnostics where false positive and false negative tests are common and they require more expensive follow up to clarify. Multi-target detection can overcome situations where the target of interest is present in low frequency and a weak positive signal can be interpreted incorrectly as a false positive. Moreover, multi-target detection can also overcome false-negative calls since it is unlikely that all targeted sequences will be missed if the gene or species is present. The generation of at least two signals in parallel is particularly valuable for diagnostic situations where the added cost of using two probes is trivial relative to the high costs associated with either retesting a patient or making the wrong treatment decision.

Thus, in another embodiment of the present invention there is provided a method for improving signaling and/or reliability in detecting a genus, a species, a gene or other related nucleic acid targets, said method comprising the steps of obtaining a biological sample from a human, an animal or an organism, contacting the biological sample with the labeled probes and flanking primer components comprising the multi-probe system as described herein, and detecting a signal emitted by the label upon contacting the target in the biological sample; where, if the labeled probes comprise an identical label, the signal is amplified, thereby improving signaling in detecting the target, or where, if the labeled probes each comprise a different label, detection is confirmed, thereby improving reliability in detecting the target; or a combination thereof. In this embodiment, the probes detect multiple target sequences sequentially or separately.

In these embodiments, the multi-probe system is configured to detect two or more related signature sequences selected from the group of genes or species consisting of: a vanA gene of vancomycin-resistant Enterococcus (VRE), a vanB gene of VRE, a Trichomonas vaginalis-specific repeated sequence (GenBank: L23861.1), an EGFR 858 mutant site, an EGFR exon 19 deletion site, a 5′UTR region of Enterovirus or Parechovirus or a combination thereof, an L1 gene of human papillomavirus 16 (HPV-16), an L1 gene of human papillomavirus 18 (HPV-18), an HA gene of influenza A or the NS1 gene of influenza B or a combination thereof, a human T-cell receptor excision circle (TREC) DNA, the Gag and Pol genes of HIV-1, and exon 4 mutant sites of the human isocitrate dehydrogenase genes IDH1 and IDH2.

In one aspect of these embodiments, the related signature sequence is the vanA gene nucleic acid sequence with GenBank Accession No. M97297.1, the system comprising a pair of flanking primers specific to the vanA coding domain sequence (CDS) in the region from about nucleotide 7316 to about nucleotide 7381 and in the region from about nucleotide 7438 to about nucleotide 7488, and/or two iDDS probe:antiprobe systems specific to the vanA CDS in the region from about nucleotide 7368 to about nucleotide 7413 and in the region from about nucleotide 7407 to about 7451, or a combination thereof. This system could thus amplify and detect two different target sites in the vanA gene of vancomycin resistant Enterococcus.

In this aspect, the primers and probes selected for amplification and/or detection of a vanA gene nucleic acid sequence (GenBank Accession No. M97297.1) may comprise: a forward primer GATATTCAAAGCTCAGCAATTTGT (SEQ ID NO: 5), a reverse primer, TAGCTGCCACCGGCCTA (SEQ ID NO: 6), a first iDDS probe, CTCCCGCCTTTTGGGTTATT AA (SEQ ID NO: 1), a first iDDS antiprobe, TTAATAACCCAAATGGCGGGAG (SEQ ID NO: 2), a second iDDS probe, CGCAACGATGTATGTCAACGA (SEQ ID NO: 3), and a second iDDS antiprobe, TCGTTGACATACCTCGTTGCG (SEQ ID NO: 4); where the probes are labeled with a fluorophore or a fluorophore and a quencher, and the antiprobes are labeled with a quencher.

In another aspect, the related signature sequence is the vanB gene nucleic acid sequence with GenBank Accession No. CP003351.1, the system comprising a pair of flanking primers specific to the vanB coding domain sequence (CDS) in the region from about nucleotide 2839810 to about nucleotide 2840049 and in the region from about nucleotide 2839810 to about nucleotide 2840049, and/or two iDDS probe:antiprobe systems specific to the vanB CDS in the region from about nucleotide 2839878 to about nucleotide 2839958 and in the region from about nucleotide 2839936 to about 2839995, or a combination thereof. This system could thus amplify and detect two different target sites in the vanB gene of vancomycin resistant Enterococcus faecium.

In this aspect of the disclosure, the primers and probes selected for amplification and/or detection of a vanB gene nucleic acid sequence (GenBank Accession No. CP003351.1) may comprise: a forward primer, CAAATCACTGGCCTACATTCTTAC (SEQ ID NO: 11), a reverse primer, CAGCGTTAAGTTCTTCCGTACC (SEQ ID NO: 12), a first iDDS probe, ATCGCCGTTC CCGAATTTC (SEQ ID NO: 7), a first iDDS antiprobe, GAAATTCGGGAACGGCCAT (SEQ ID NO: 8), a second iDDS probe, CGCCTCCGGCTTGTCAC (SEQ ID NO: 9), and a second iDDS antiprobe, GTGACAAGCCGGTGGCG (SEQ ID NO: 10); where the probes are labeled with a fluorophore or a fluorophore and a quencher, and the antiprobes are labeled with a quencher.

In yet another aspect of the disclosure, the related signature sequence is the Trichomonas vaginalis (T. vag.) repetitive nucleic acid sequence with GenBank Accession No. L23861.1, the system comprising a pair of flanking primers specific to the T. vag. sequence in the region from about nucleotide 580 to about nucleotide 639 and in the region from about nucleotide 678 to about nucleotide 737, and/or two iDDS probe:antiprobe systems specific to the T. vag. sequence in the region from about nucleotide 623 to about nucleotide 659 and in the region from about nucleotide 645 to about 686, or a combination thereof. This system could thus amplify and detect two different target sites in the repetitive nucleic acid sequence of T. vaginalis.

In this aspect of the disclosure, the primers and probes selected for amplification and/or detection of the T. vag. repetitive nucleic acid sequence (GenBank Accession number L23861.1) may comprise: a forward primer, AATTTCCGTTTAATTTCATGGTC (SEQ ID NO: 17), a reverse primer, TTYGTGTCTCGTGCCATAGTC (SEQ ID NO: 18), a first iDDS probe, TCCTCGGAGTCTTTGAATCGG (SEQ ID NO: 13), a first iDDS antiprobe, CCGATTCATAGAC TCCGAGG (SEQ ID NO: 14), a second iDDS probe, ACCAAGAATGGTGTAACTCGACCT (SEQ ID NO: 15), and a second iDDS antiprobe, AGGTAGAGTTACACCATTCTTGGT (SEQ ID NO: 16); where the probes are labeled with a fluorophore or a fluorophore and a quencher, and the antiprobes are labeled with a quencher.

In yet another aspect, the related signature sequence is the wild type (WT) EGFR nucleic acid sequence or the L858R mutant EGFR nucleic acid sequence with GenBank Accession No. NW_{—}004078029.1 or both, the system comprising a pair of flanking primers specific to the EGFR DNA sequence in the region from about nucleotide 55207745 to about nucleotide 55207805 and in the region from about nucleotide 55207824 to about nucleotide 55207884, and/or two iDDS probe:antiprobe systems specific to the WT or mutant EGFR sequence in the region from about nucleotide 55207792 to about 55207832, or a combination thereof. This system could thus amplify and detect both the WT human EGFR and the L858R EGFR mutant.

In this aspect, the primers and probes selected for amplification and/or detection of a human WT and/or L858R mutant EGFR nucleic acid sequence (GenBank Accession No. NM_{—}005228.3) comprise: a forward primer, GAAAACACCGCAGCATGTC (SEQ ID NO: 23), a reverse primer, CTGCATGGTATTCTTTCTCTTCC (SEQ ID NO: 24), a WT iDDS probe, AGCA GTTTGGGCAGCCCAA (SEQ ID NO: 19), a WT iDDS antiprobe, TTGGGCTGGCCTAACTGCT (SEQ ID NO: 20), a mutant iDDS probe, AGCAGTTTGGGCCGCCC, (SEQ ID NO: 21), and a mutant iDDS antiprobe GGGCGGGCCAAACGGCT (SEQ ID NO: 22); where the probes are labeled with a fluorophore or a fluorophore and a quencher, the antiprobes are labeled with a quencher. The iDDS probes optionally may have one or more synthetic bases with enhanced binding, such as LNAs or BNAs.

In yet another aspect, the related signature sequences are a variable exon 19 deletion mutant sequence and an adjacent reference sequence in the human EGFR gene with GenBank Accession number NW_{—}004078029.1), said system comprising a pair of flanking primers specific to the EGFR sequence in the region from about nucleotide 55242321 to about nucleotide 55242414 and about 55242489-55242537, and/or two iDDS probe:antiprobes specific to an EGFR WT or deletion mutant target sequence in the region from about nucleotide 55242412 to about nucleotide 55242444 and from about nucleotide 55242458 to about nucleotide 55242489, respectively. This system could thus amplify and detect both the reference sequence and the EGFR deletion mutant.

In this aspect, the primers and probes selected for amplification and/or detection of a human WT EGFR and/or EGFR deletion mutant sequence (GenBank Accession No. NW_{—}004078029.1) comprise: a forward primer, GGTAACATCCACCCAGATCA (SEQ ID NO: 29), a reverse primer, CATCGAGGATTTCCTTGTTG (SEQ ID NO: 30), a first iDDS probe, CT CTGGATCCCAGAAGGTGAGA (SEQ ID NO: 25), a first iDDS antiprobe, TCTCTCCTTCTGGG ATCCAGAG (SEQ ID NO: 26), a second iDDS probe, CAAGGAATTAAGAGAAGCAACATC (SEQ ID NO: 27), and a second iDDS antiprobe, GATGTTGCCTCTCTTAATTCCTTG (SEQ ID NO: 28); where the probes are labeled with a fluorophore or a fluorophore and a quencher, and the antiprobes are labeled with a quencher.

In yet another aspect, the related signature sequence is a 5′-untranslated region of the enterovirus (EV) sequence with GenBank Accession No. HQ728259.1 or a parechovirus (PV) nucleic acid sequence with GenBank Accession No. EF051629.2, said system comprising a pair of flanking primers specific to EV DNA in the region from about nucleotide 446 to about nucleotide 487 and from about nucleotide 583 to about nucleotide 642, a pair of flanking primers specific to PV DNA in the region from about nucleotide 420 to about nucleotide 461 and from about nucleotide 641 to about nucleotide 680, and/or two iDDS probe:antiprobe systems specific to the EV sequence in the region from about nucleotide 527 to about nucleotide 603, or two iDDS probe:antiprobe systems specific to the PV sequence in the region from about nucleotide 530 to about nucleotide 590 and from about nucleotide 556 to about nucleotide 616, or combinations thereof. This system could thus amplify and detect two different target sites in the both the EV and PV 5′UTRs.

In this aspect, the primers and probes selected for amplification and/or detection of 5′UTR of EV or PV nucleic acid sequences (GenBank Accession Nos. HQ728259.1 and EF051629.2, respectively) comprise: a forward EV primer, CCCCTGAATGCGGMTAAT (SEQ ID NO: 35), a reverse EV primer, RATTGTCACCATAAGCAGCCA (SEQ ID NO: 36), a first EV iDDS probe, CGGAACCGACTACTTTGGGTG (SEQ ID NO: 31), a first EV iDDS antiprobe, CA CCCAATG TAGTCGGTTCCG (SEQ ID NO: 32), a second EV iDDS probe, AAACACGGACAC CCAAAGTAGTCG (SEQ ID NO: 33), a second EV iDDS antiprobe, CGACTACTTTGGTTGTC CGTGTTT (SEQ ID NO: 34), a PV forward primer, GGCCAAAAGCCAAGGTTTA (SEQ ID NO: 41), a PV reverse primer, TTGGCCCACTAGACGTTTTT (SEQ ID NO: 42), a first PV iDDS probe, CACTATGGATCTGATCTGGGGC (SEQ ID NO: 37), a first PV iDDS antiprobe, GCCCC AGATCAGATCCTTAGTG (SEQ ID NO: 38), a second PV iDDS probe, CGGGTACCTTCTGG GCATC (SEQ ID NO: 39), and a second PV iDDS antiprobe, GATGCCCTGAAGGTACCCG (SEQ ID NO: 40); where the probes are labeled with a fluorophore or a fluorophore and a quencher, and the antiprobes are labeled with a quencher.

In yet another aspect, the related signature sequence is a human papillomavirus 16 (HPV16) L1 gene nucleic acid sequence with GenBank Accession No. EU430688.1), comprising a pair of flanking primers specific to the L1 CDS in the nucleotide region from about nucleotide 938 to about nucleotide 1020 and from about nucleotide 1077 to about nucleotide 1155, and/or two iDDS probe:antiprobe systems specific to the L1 CDS in the region from about nucleotide 1020 to about nucleotide 1050 and from about nucleotide 1032 to about nucleotide 1063, or a combination thereof. This system could thus amplify and detect two different target sites in the HPV-16 L1 gene.

In this aspect, the primers and probes selected for amplification and/or detection of an HPV16 L1 gene nucleic acid sequence (GenBank Accession No. EU430688.1) comprise: a forward primer, GGTTACCTCTGATGCCCAAATA (SEQ ID NO: 47), a reverse primer, TTTCTGAAGTAGATATGGCAGCAC (SEQ ID NO: 48), a first iDDS probe, CGAGCACAGGGC CACAAT (SEQ ID NO: 43), a first iDDS antiprobe, ATTGTGGTCCTGTGCTCG (SEQ ID NO: 44), a second iDDS probe, GGTTA CCCCAACAAATGCCA (SEQ ID NO: 45), and a second iDDS antiprobe, TGGCTTTTGTTGGGGTAACC (SEQ ID NO: 46); wherein the probes are labeled with a fluorophore or a fluorophore and a quencher, and the antiprobes are labeled with a quencher.

In yet another aspect, the related signature sequence is a human papillomavirus 18 (HPV18) L1 gene nucleic acid sequence with GenBank Accession No. EU834744.1, comprising a pair of flanking primers specific to the HPV18 CDS in the region from about nucleotide 1032 to about nucleotide 1103 and from about nucleotide 1196 to about nucleotide 1287, and/or two iDDS probe:antiprobe systems specific to the HPV18 CDS in the region from about nucleotide 1098 to about nucleotide 1160 and from about nucleotide 1125 to about nucleotide 1185, or a combination thereof. This system could thus amplify and detect two different target sites in the HPV-18 L1 gene.

In this aspect, the primers and probes selected for amplification and/or detection of an HPV18 L1 gene nucleic acid sequence (GenBank Accession number EU834744.1) comprise: a forward primer, TGTTACCTCTGACTCCCAGTTG (SEQ ID NO: 53), a reverse primer, TTGCC CAGGTACAGGAGACT (SEQ ID NO: 54), a first iDDS probe, GGTTACATAAGGCACAGGGTC ATAAC (SEQ ID NO: 49), a first iDDS antiprobe, GTTATGACCCTGTCCCTTATGTAACC (SEQ ID NO: 50), a second iDDS probe, TTGATTATGCCAGCAAACACCA (SEQ ID NO: 51), and a second iDDS antiprobe, TGGAGTT TGCTGGCATAATCAA (SEQ ID NO: 52); where the probes are labeled with a fluorophore a fluorophore and a quencher, and the antiprobes are labeled with a quencher.

In yet another aspect, the related signature sequence is an influenza A or B nucleic acid sequence, said system comprising four sets of flanking primers and associated iDDS probe:antiprobes, comprising (i) a first set of a pair of flanking primers specific to the hemagglutinin (HA) CDS of an H1N1 seasonal influenza A with GenBank Accession No. CY069381.1 in the region from about nucleotide 25 to about nucleotide 95 and from about nucleotide 225 to about nucleotide 295, and an iDDS probe:antiprobe system specific to the HA CDS in the region from about nucleotide 185 to about nucleotide 245; (ii) a second set of a pair of flanking primers specific to the HA CDS of a 2009 H1 N1 pandemic influenza A with GenBank Accession No. FJ969540.1 in the region from about nucleotide 878 to about nucleotide 938 and from about nucleotide 971 to about nucleotide 1061, and an iDDS probe:antiprobe system specific to the HA CDS in the region from about nucleotide 920 to about nucleotide 980; (iii) a third set of a pair of flanking primers specific to the HA CDS of an H3N2 influenza A with GenBank Accession No. CY120904 in the region from about nucleotide 285 to about nucleotide 345 and from about nucleotide 370 to about nucleotide 430, and an iDDS probe:antiprobe system specific to the HA CDS in the region from about nucleotide 329 to about nucleotide 389; and (iv) a fourth set of a pair of flanking primers specific to the NS1 CDS of an influenza B with GenBank Accession No. JQ034241.1 in the region from about nucleotide 770 to about nucleotide 830 and from about nucleotide 892 to about nucleotide 952, and an iDDS probe:antiprobe system specific to the HA CDS in the nucleotide region from about nucleotide 831 to about nucleotide 891. This system would thus amplify and detect different target sites in the HA gene of H3N2 and seasonal and 2009-pandemic H1N1 influenza A strains, and the NS1 gene of influenza B strains.

In this aspect, the primers and probes selected for amplification and/or detection of influenza A or B nucleic acid sequences comprise: the first set forward primer, GCTACCATGC TAACAACTCGC (SEQ ID NO: 57), the first set reverse primer, TCGCATTCTGGGTTTCC (SEQ ID NO: 58), the first set iDDS probe, TTGGGTAACTGCAGCGTTGC (SEQ ID NO: 55), the first set iDDS antiprobe, GCA ACGCAGCAGTTACCCAA (SEQ ID NO: 56), the second set forward primer, ATAAACACCAGCCTC CCATT (SEQ ID NO: 61), the second set reverse primer, GT G GCCAGTCTCAATTTTGT (SEQ ID NO: 62), the second set iDDS probe, TCCGATCACAATTG GAAAATGTC (SEQ ID NO: 59), the second set iDDS antiprobe, GACTTTTTCCAATTGTGATC GGA (SEQ ID NO: 60), the third set forward primer, CCTTTTTGTTGAACGAAGCA (SEQ ID NO: 65), the third set reverse primer, TCATT GTTAAACTCCAGTGTGC (SEQ ID NO: 66), the third set iDDS probe, TATGATGTGCCGGATTAT GCCT (SEQ ID NO: 63), the third set iDDS antiprobe, AGGCATAATCCGGCTCATCATA (SEQ ID NO: 64), the fourth set forward primer, GTCAAGAGCACCGATTATCAC (SEQ ID NO: 69), the fourth set reverse primer, CAACCATGT CAGCTATTATGGAG (SEQ ID NO: 70), the fourth set iDDS probe, CCGTGACCAGTCTAATT GTCTCC (SEQ ID NO: 67), and the fourth set iDDS antiprobe, GGAGACATTTAGACTGGTCA CGG (SEQ ID NO: 68); where the probes are labeled with a fluorophore or a fluorophore and a quencher, and the antiprobes are labeled with a quencher.

In yet another aspect, the related signature sequence is the T-cell receptor excision circle (TREC) nucleic acid sequences derived from excision of Human Chromosome 14 DNA with GenBank Accession No. NW_{—}004078079.1, said system comprising a pair of flanking primers specific to the TREC sequence in the region from about nucleotide 3941495 to about nucleotide 3941555 and from about nucleotide 3853115 to about nucleotide 3853175, two iDDS probe:antiprobe systems specific to the TREC sequence in the region from about nucleotide 3941545 to about nucleotide 3941580 and from about nucleotide 3853091 to about nucleotide 3853120, or a combination thereof. This system would thus amplify and detect two different target sites in TREC DNA.

In this aspect, the primers and probes selected for amplification and/or detection of TREC nucleic acid sequences derived from excision of Human Chromosome 14 DNA (GenBank Accession number NW_{—}004078079.1) comprise: a forward primer, TTCAACCATGCTGACACC TC (SEQ ID NO: 75), a reverse primer, CAGCTGCAGGGTTTAGGC (SEQ ID NO: 76), a first iDDS probe, TTGTAAAGGTGCCCACTCCTGT (SEQ ID NO: 71), a first iDDS antiprobe, ACAG GAGTGGGCTCCTTTACAA (SEQ ID NO: 72), a second iDDS probe, TGCAGGTGCCTATGCA TCAC (SEQ ID NO: 73), and a second iDDS antiprobe, GTGATGCTTAGGCACCTGCA (SEQ ID NO: 74); where the probes are labeled with a fluorophore or a fluorophore and a quencher, and the antiprobes are labeled with a quencher.

In yet another aspect, the related signature sequences are a human immunodeficiency virus (HIV) Gag nucleic acid sequence with GenBank Accession No. JX244938.1 and a human immunodeficiency virus (HIV) Pol gene nucleic acid sequence with GenBank Accession No. KC462191.1, said system comprising a flanking primer specific to the Gag CDS in the region from about nucleotide 790 to about nucleotide 850, a first Half-Universal probe:antiprobe system specific to the Gag CDS in the region from about nucleotide 729 to about nucleotide 789, a flanking primer specific to the Pol CDS in the region from about nucleotide 1423 to about nucleotide 1483, and a second Half-Universal probe:antiprobe system specific to the Pol CDS in the region from about nucleotide 1305 to about nucleotide 1365. This system would thus amplify and detect HIV at target sites in both the Gag and Pol genes.

In this aspect, the primers and probes selected for amplification and/or detection of human immunodeficiency virus (HIV) Gag and Pol gene nucleic acid sequences (GenBank Accession Nos. JX244938.1 and KC462191.1, respectively) comprise: a Gag primer, ATGCTG ACAGGGCTATACATTCTT (SEQ ID NO: 79), a Pol primer, AGGGGTCGTTGCCAAAGAGTGA T (SEQ ID NO: 81), a Gag Half-Universal probe, CTACGTAGACTAGACGTTCACCTATCCCA GTGGGAGAGAT (SEQ ID NO: 77), a Pol Half-Universal probe, CTACGTAGACTAGACGTTC CAGAGCAGACCAGAGCCA (SEQ ID NO: 80), and a Half-Universal antiprobe, GAACGTCTA GTCTACGTAG (SEQ ID NO: 78); where the probes are labeled with a fluorophore or a fluorophore and a quencher, and the antiprobes are labeled with a quencher.

In yet another aspect, the related signature sequences are point mutations in the human IDH1 with GenBank Accession No. NT_{—}005403.17 or IDH2 gene with GenBank Accession No. NC_{—}000015.9, said system comprising a pair of flanking primers specific to IDH1 DNA in the region from about nucleotide 59322435 to about nucleotide 59322519 and from about nucleotide 59322545 to about nucleotide 59322614, and a pair of flanking primers specific to IDH2 DNA in the region from about nucleotide 90631711 to about nucleotide 90631770 and from about nucleotide 90631981 to about 90632040, or two iDDS probe:antiprobe systems specific to mutations in the IDH1 sequence in the region from about nucleotide 59322510 to about nucleotide 59322550 or in the IDH2 sequence from about nucleotide 90631818 to about nucleotide 90631857, or combinations thereof. This system could thus amplify and detect two target sites in the IDH1 and IDH2 genes.

In this aspect, the primers and probes selected for amplification and/or detection of two point mutations in either the human IDH1 or IDH2 gene (GenBank Accession Nos. NT_{—}005403.17 and NC_{—}000015.9, respectively) comprise: a forward IDH1 primer, GGCTTGTG AGTGGATGGGTA (SEQ ID NO: 86), a reverse IDH1 primer, AAAACATGCAAAATCACATTAT (SEQ ID NO: 87), a first mutant IDH1 iDDS probe, CATAGGTCATGATGCTTATGGGGA (SEQ ID NO: 82), a first mutant IDH1 iDDS antiprobe, TCCGCATAAGCATGATGACCTATG (SEQ ID NO: 83), a second mutant IDH1 iDDS probe, CCATAAGCATGACAAGCTATGATG (SEQ ID NO: 84), a second mutant IDH1 iDDS antiprobe, CATCATAGGTTGTCTTGCTTATGG (SEQ ID NO: 85), a forward IDH2 primer, CACTATTATCTCTGTCCTCA (SEQ ID NO: 92), a reverse IDH2 primer, AGACAAGAGGATGGCTA (SEQ ID NO: 93), a first mutant IDH2 iDDS probe, CC ATTGGCAAGGACGCCC (SEQ ID NO: 88), a first mutant IDH2 iDDS antiprobe, GGGCGTGCT TGCCTATGG (SEQ ID NO: 89), a second mutant IDH2 iDDS probe, CCATTGGCATGGACGC CC (SEQ ID NO: 90), and a second mutant IDH2 iDDS antiprobe, GGGCGTGCATGCCTATGG (SEQ ID NO: 91); where the probes are labeled with a fluorophore or a fluorophore and a quencher, the antiprobes are labeled with a quencher. The mutant IDH probes optionally have one or more enhanced-binding nucleotides, such as LNAs or BNAs.

In yet another aspect, at least one related signature sequence is selected from the group of genes or species consisting of (1) exon 2, codon 12 or codon 13 or both mutant sites of a KRAS gene with GenBank Accession No. NM_{—}004985, (2) a codon 600 mutant site of the BRAF gene with GenBank Accession No. NM_{—}004333.4), (3) a Pseudomonas aeruginosa algD gene with GenBank Accession No. CP000438), (4) a Burkholderia cepacia complex recA gene with GenBank Accession No. NC_{—}018513.1), (5) a Stenotrophomonas maltophilia 23S gene with GenBank Accession No. NC_{—}015947), (6) an Achromobacter species OXA-114-like gene with GenBank Accession No. EU188842.1), (7) a Staphylococcus aureus femA gene with GenBank Accession No. CP003194), (8) a Staphylococcus aureus mecA gene with GenBank Accession No. AB033763.2), (9) a Mycobacterium avium complex intergenic spacer gene with GenBank Accession No. L07855.2) or Mycobacterium chelonae with GenBank Accession No. DQ866771.1 or both, and (10) a Mycobacterium abscessus (GenBank: HG313848.1) (MCAG) intergenic spacer gene.

In this aspect, the related signature sequences are a reference sequence and an adjacent mutant region in exon 2, codon 12 or 13 of the human KRAS gene, said system comprising a flanking primer specific to the KRAS sequence in the region from about nucleotide 144 to about nucleotide 204, a ZIPR reference probe specific to the WT KRAS sequence in the region from about nucleotide 231 to about nucleotide 291, and a WT iDDS probe specific to a presumptive mutant sequence in codon 12 or codon 13 in the region from about nucleotide 205 to about nucleotide 230. This system would thus amplify and detect KRAS amplicons using the reference ZIPR probe, and assess the relative frequency of single base mutants in the mutant region with the WT iDDS probe.

Also in this aspect, the primers and probes selected for amplification and/or detection of a reference sequence and an adjacent mutant region in exon 2, codon 12 or 13 of the human KRAS gene (GenBank Accession No. NM_{—}004985) comprise: a forward primer, CCTGCTGAA AATGACTGAATATAAA (SEQ ID NO:94), a iDDS probe, CCTACGCCACCAGCTC (SEQ ID NO: 95), a iDDS antiprobe, GAGGTGGTGGCGTAGG (SEQ ID NO: 96), a ZIPR probe, TGGAT CATATTCGTCCACAAAA (SEQ ID NO: 97), and a ZIPR antiprobe, TTTTGAGGACGAATATGA TCCA (SEQ ID NO: 98); where the iDDS probe is labeled with a fluorophore or a fluorophore and a quencher, the iDDS antiprobe is labeled with a quencher, the ZIPR probe is labeled with a fluorophore, and the ZIPR antiprobe is labeled with a quencher. The iDDS probe optionally has one or more synthetic nucleotides with enhanced binding, such as LNAs or BNAs.

In yet another aspect, the related signature sequence is a mutant site in codon 600 of the human BRAF gene or a WT reference sequence of BRAF or both, said system comprising a pair of flanking primers specific to the BRAF sequence in the region from about nucleotide 1785 to about nucleotide 1844 and from about nucleotide 1876 to about nucleotide 1935, a V600E mutant iDDS probe, and a WT iDDS probe in the region from about nucleotide 1845 to about nucleotide 1875. This system would thus detect all amplicons using the WT iDDS probe, and assess the relative frequency of the V600E mutant with the V600E mutant iDDS probe.

In this aspect, the primers and probes selected for amplification and/or detection of a WT sequence and an adjacent mutant region in codon 600 of the human BRAF gene (GenBank Accession No. NM_{—}004985) comprise: the primers, TGAAGACCTCACAGTAAAAATAGGTG (SEQ ID NO: 99 97) and CCACAAAATGGATCCAGACA (SEQ ID NO: 100), the mutant iDDS probe, AGCTACTGAGAAATCTCGATGG (SEQ ID NO: 101), the mutant iDDS antiprobe, CATCGAG ATTTCTGTGTAGCT (SEQ ID NO: 102), the WT iDDS probe, CAACTCATCACGGAGCTCATG (SEQ ID NO: 103), and the WT iDDS antiprobe, CATGAGCTGCGTGATGTGTTG (SEQ ID NO: 104); where the probes are labeled with a fluorophore or a fluorophore and a quencher and the antiprobes are labeled with a quencher. The iDDS probes optionally have one or more synthetic nucleotides with enhanced binding, such as LNAs or BNAs.

In yet another aspect, the related signature sequence is at least one bacterial species commonly associated with cystic fibrosis, said system comprising eight sets of flanking primers or associated iDDS probe:antiprobe systems or both, comprising (i) a first set of flanking primers specific to the Pseudomonas aeruginosa algD gene with GenBank Accession No. CP000438.1 in the region from about nucleotide 1595235 to about nucleotide 1595294 and from about nucleotide 1595355 to about nucleotide 1595414, or an iDDS probe:antiprobe system specific to the Pseudomonas aeruginosa algD gene in the region from about nucleotide 1595295 to about nucleotide 1595354 or a combination thereof; (ii) a second set of flanking primers specific to the Burkholderia cepacia complex recA gene with GenBank Accession No. NC_{—}018513.1 in the region from about nucleotide 869329 to about nucleotide 869388 and from about nucleotide 869460 to about nucleotide 869519 or an iDDS probe:antiprobe system specific to the Burkholderia cepacia complex recA gene in the region from about nucleotide 869389 to about nucleotide 869448 or a combination thereof; (iii) a third set of flanking primers specific to the Stenotrophomonas maltophilia 23S gene with GenBank Accession No. NC_{—}015947 in the region from about nucleotide 368121 to about nucleotide 368180 and from about nucleotide 368216 to about nucleotide 368275, or an iDDS probe:antiprobe system specific to the Stenotrophomonas maltophilia 23S gene in the region from about nucleotide 368179 to about nucleotide 368215, or a combination thereof; (iv) a fourth set of flanking primers specific to the Achromobacter species OXA-114-like gene with GenBank Accession No. EU188842.1 in the region from about nucleotide 230 to about nucleotide 289 and from about nucleotide 350 to about nucleotide 409, or an iDDS probe:antiprobe system specific to the Achromobacter species OXA-114-like gene in the nucleotide region from about nucleotide 290 to about nucleotide 349 or a combination thereof; (v) a fifth set of flanking primers specific to the Staphylococcus aureus femA gene with GenBank Accession No. CP003194.1 in the region from about nucleotide 1410168 to about nucleotide 1410227 and from about nucleotide 1410228 to about nucleotide 1410347, or an iDDS probe:antiprobe system specific to the Staphylococcus aureus femA gene in the region from about nucleotide 1410228 to about nucleotide 1410287, or a combination thereof; (vi) a sixth set of flanking primers specific to the Staphylococcus aureus mecA gene with GenBank Accession No. AB033763.2 in the region from about nucleotide 31680 to about nucleotide 31739 and from about nucleotide 31800 to about nucleotide 31859, or an iDDS probe:antiprobe system specific to the Staphylococcus aureus mecA gene in the nucleotide region from about nucleotide 31740 to about nucleotide 31799, or a combination thereof; (vii) a seventh set of flanking primers specific to the Mycobacterium avium complex intergenic spacer gene with GenBank Accession No. L07855.2 in the region from about nucleotide 15 to about nucleotide 74 and from about nucleotide 135 to about nucleotide 194, or an iDDS probe:antiprobe system specific to the Mycobacterium avium complex intergenic spacer gene in the region from about nucleotide 75 to about nucleotide 134, or a combination thereof; and (viii) an eighth set of common flanking primers specific to both the Mycobacterium chelonae intergenic spacer gene with GenBank Accession No. DQ866771.1 in the region from about nucleotide 1461 to about nucleotide 1520 and from about nucleotide 1571 to about nucleotide 1630 and the Mycobacterium abscessus intergenic spacer gene with GenBank Accession No. HG313848.1 in the region from about nucleotide 26 to about nucleotide 85 and from about nucleotide 146 to about nucleotide 205, or an iDDS probe:antiprobe system specific to the Mycobacterium chelonae intergenic spacer gene in the region from about nucleotide 1521 to about nucleotide 1570, or an iDDS probe:antiprobe system specific to the Mycobacterium abscessus intergenic spacer gene in the region from about nucleotide 86 to about nucleotide 145, or combinations thereof. This system would thus detect one or more bacterial species commonly associated with cystic fibrosis.

In another aspect of the disclosure, the primers and probes selected for amplification and/or detection of at least one bacterial species commonly associated with cystic fibrosis comprise: the first set forward primer, CGGGGCTCGTAGTAACG (SEQ ID NO: 105), the first set reverse primer, CGACATAGCCCAAACCAAA (SEQ ID NO: 106), the first set iDDS probe, TGAATGCGATGCGAATCAGC (SEQ ID NO: 107), the first set iDDS antiprobe, GCTGATTCG CTTCGCATTCA (SEQ ID NO: 108), the second set forward primer, GGCTCGATCAAGAAGAA CGA (SEQ ID NO: 109), the second set reverse primer, GACGATCTTCGCCTGCAC (SEQ ID NO: 110), the second set iDDS probe, CGTTCCGCGAAGCGATCT (SEQ ID NO: 111), the second set iDDS antiprobe, AGATTGCTTCGCGGAACG (SEQ ID NO: 112), the third set forward primer, CAGAGYGTTTTCCTAGAACCA (SEQ ID NO: 113), the third set reverse primer, GTATCCTGCAGTGAATACATAGCTG (SEQ ID NO: 114), the third set iDDS probe, AC CAGGTTCGCTTCCAGCA (SEQ ID NO: 115), the third set iDDS antiprobe, TGCTGGTAGCGA ACCTGGT (SEQ ID NO: 116), the fourth set forward primer, GAGCCGGTCTGGAACTACC (SEQ ID NO: 117), the fourth set reverse primer, TGGTGTAGCGCTGGAAGC (SEQ ID NO: 118 116), the fourth set iDDS probe, CGCTGGATCAAGTATTCGGTGG (SEQ ID NO: 119), the fourth set iDDS antiprobe, CCTCCGAATACTTGATCCAGCG (SEQ ID NO: 120), the fifth set forward primer, ACAGATAGCATGCCATACAGTCA (SEQ ID NO: 121), the fifth set reverse primer, GCAGAAGTAAGCAAGCTGCAA (SEQ ID NO: 122), the fifth set iDDS probe, CACGCA AACTGTTGGCCACT (SEQ ID NO: 123), the fifth set iDDS antiprobe, AGTGGCTAACAGTTTG CGTG (SEQ ID NO: 124), the sixth set forward primer, CTACGGTAACATTGATCGCAAC (SEQ ID NO: 125), the sixth set reverse primer, TT TCAATATGTATGCTTTGGTCTTTC (SEQ ID NO: 126), the sixth set iDDS probe, TGCATCCTGGAATAATGACGC (SEQ ID NO: 127), the sixth set iDDS antiprobe, GCGTCCTTATTCCAGGAATGCA (SEQ ID NO: 128), the seventh set forward primer, GGAGCACCACGAAAAGCA (SEQ ID NO: 129), the seventh set reverse primer, ATGGAGGGACTCCACACG (SEQ ID NO: 130), the seventh set iDDS probe, CAACAGCAAAT GATTGCCAGAC (SEQ ID NO: 131), the seventh set iDDS antiprobe, GTCTGGAAATCATTTG CTGTTG (SEQ ID NO: 132), the eighth set forward primer, CTTTCTAAGGAGCACCATTTCC (SEQ ID NO: 133), the eighth set reverse primer, YATCCACGGGGTGGACAG (SEQ ID NO: 134), the eighth set Mycobacterium chelonae iDDS probe, TCTGTAGTGGTTACTCGCTTGGTG (SEQ ID NO: 135), the eighth set Mycobacterium chelonae iDDS antiprobe, CTTCAAGCGAGT AACC ACTACAGA (SEQ ID NO: 136), the eighth set Mycobacterium abscessus iDDS probe, GAATGAACTAGGGAACATAAAGTAGGCA (SEQ ID NO: 137), and the eighth set Mycobacterium abscessus iDDS antiprobe, TGCCTACTTTTAGTTCCCTAGTTCATTC (SEQ ID NO: 138); wherein the probes are labeled with a fluorophore and optionally a quencher, and the antiprobes are labeled with a quencher.

In yet another aspect the two or more related signature sequences are in the same gene or species and wherein the probe technology is selected from the group consisting of iDDS probes, iDDS-2Q probes and MacMan probes, said system configured for endpoint detection with simultaneous confirmation thereof.

The following examples are put forth to provide those of ordinary skill in the art with a complete disclosure and description of how to perform the methods and use the compositions and compounds disclosed and claimed herein. Temperature is in ° C.

### EXAMPLE 1

### Real-time PCR Using iDDS Probes for the Detection of Vancomycin A (VA)-resistant Enterococcus (vanA VRE)

Each tube contained either an ULTRAMER® oligonucleotide template comprising the targeted vanA gene segment, or DNA extracted from clinical samples. The probes, antiprobes, and primers were fabricated with the following sequences and labeling, and were used at the indicated final concentrations:

VA probe 1:
(SEQ ID NO: 1)
FAM-CTCCCGCCTTTTGGGTTATTAA-BHQ-1
at 200 nM,
VA antiprobe 1:
(SEQ ID NO: 2)
TTAATAACCCAAATGGCGGGAG-BHQ-1
at 150 nM,
VA probe 2:
(SEQ ID NO: 3)
CFR610-CGCAACGATGTATGTCAACGA-BHQ-2
at 200 nM,
VA antiprobe 2:
(SEQ ID NO: 4)
TCGTTGACATACCTCGTTGCG-BHQ-2
at 150 nM,
VA forward primer:
(SEQ ID NO: 5)
GATATTCAAAGCTCAGCAATTTGT
at 300 nM,
and
VA reverse primer:
(SEQ ID NO: 6)
TAGCTGCCACCGGCCTA
at 300 nM.

Cycling conditions: Real-time PCR was performed with an Mx3005p instrument (Stratagene, Inc.) using HOTSTART-IT PROBE® qPCR master mix (USB, Inc.) (2×) supplemented with 1 μl of 25 mM MgCl₂ in a 20-μl reaction. To initiate hot start conditions, the tube was heated to 95° C. for 5 min followed by 60 cycles of two-step PCR (denaturation at 95° C. for 15 sec, annealing/extension at 60° C. for 1 min).

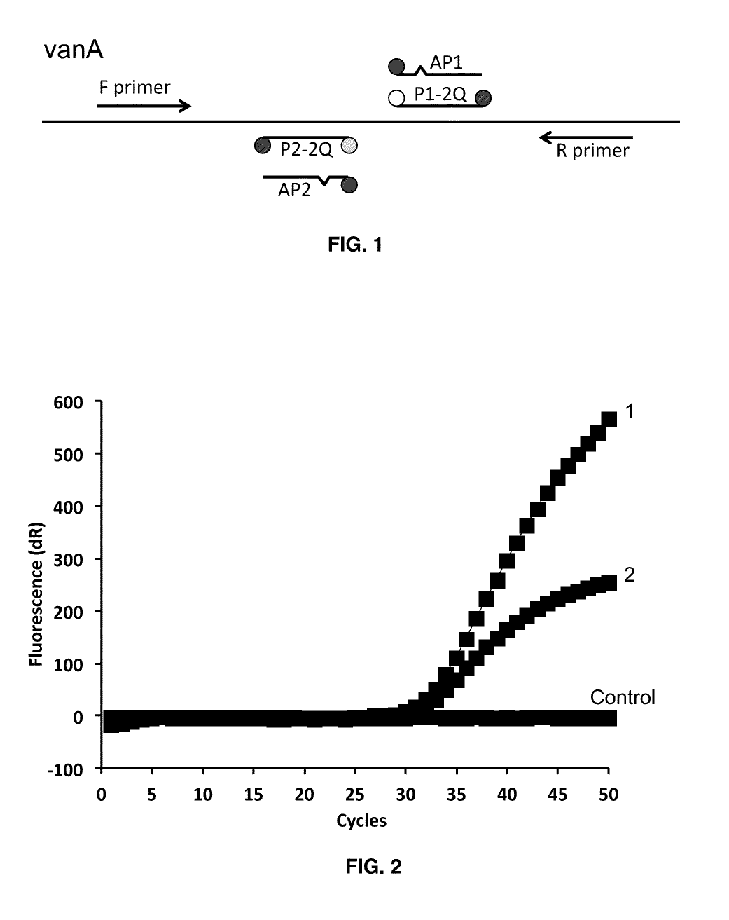

The scheme of this method is shown in FIG. 1 and the qPCR results are as expected and are shown and discussed in FIG. 2.

EXAMPLE 2

### Real-time PCR Using iDDS Probes for the Detection of Vancomycin B (VB)-resistant Enterococcus (vanB VRE)

Each tube contains an ULTRAMER® oligonucleotide template comprising the targeted vanB gene segment. The probes, antiprobes, and primers are fabricated with the following sequences and labeling, and are used at the indicated final concentrations:

VB probe 1:
(SEQ ID NO: 7)
FAM-ATCGCCGTTCCCGAATTTC
at 200 nM,
VB antiprobe 1:
(SEQ ID NO: 8)
GAAATTCGGGAACGGCCAT-BHQ-1
at 400 nM,
VB probe 2:
(SEQ ID NO: 9)
CFR610-CGCCTCCGGCTTGTCAC
at 200 nM,
VB antiprobe 2:
(SEQ ID NO: 10)
GTGACAAGCCGGTGGCG-BHQ-2
at 400 nM,
VB forward primer:
(SEQ ID NO: 11)
CAAATCACTGGCCTACATTCTTAC
at 200 nM,
and
VB reverse primer:
(SEQ ID NO: 12)
CAGCGTTAAGTTCTTCCGTACC
at 200 nM.

Cycling conditions: Real-time PCR is performed as described in Example 1, except that an annealing/extension step at 58° C. for 1 min is used during 50 cycles of 2-step PCR. The qPCR results are as expected and are shown and discussed in FIG. 2.

### EXAMPLE 3

### Real-time PCR Using iDDS Probes for the Detection of Trichomonas vaginalis (T. vag.) Repetitive DNA

Each tube contained either an ULTRAMER® oligonucleotide template comprising the targeted T. vaginalis gene segment, or DNA extracted from clinical samples. The probes, antiprobes, and primers were fabricated with the following sequences and labeling, and were used at the indicated final concentrations:

T. vag. probe 1:
(SEQ ID NO: 13)
TET-CCTCGGAGTCTTTGAATCGG-BHQ-2
at 200 nM,
T. vag. antiprobe 1:
(SEQ ID NO: 14)
CCGATTCATAGACTCCGAGG-BHQ-2
at 100 nM,
T. vag. probe 2:
(SEQ ID NO: 15)
ROX-ACCAAGAATGGTGTAACTCGACCT-BHQ-2
at 200 nM,
T. vag. antiprobe 2:
(SEQ ID NO: 16)
AGGTAGAGTTACACCATTCTTGGT-BHQ-2
at 100 nM,
T. vag. forward primer:
(SEQ ID NO: 17)
AATTTCCGTTTAATTTCATGGTC
at 300 nM,
and
T. vag. reverse primer:
(SEQ ID NO: 18)
TTYGTGTCTCGTGCCATAGTC
at 300 nM.

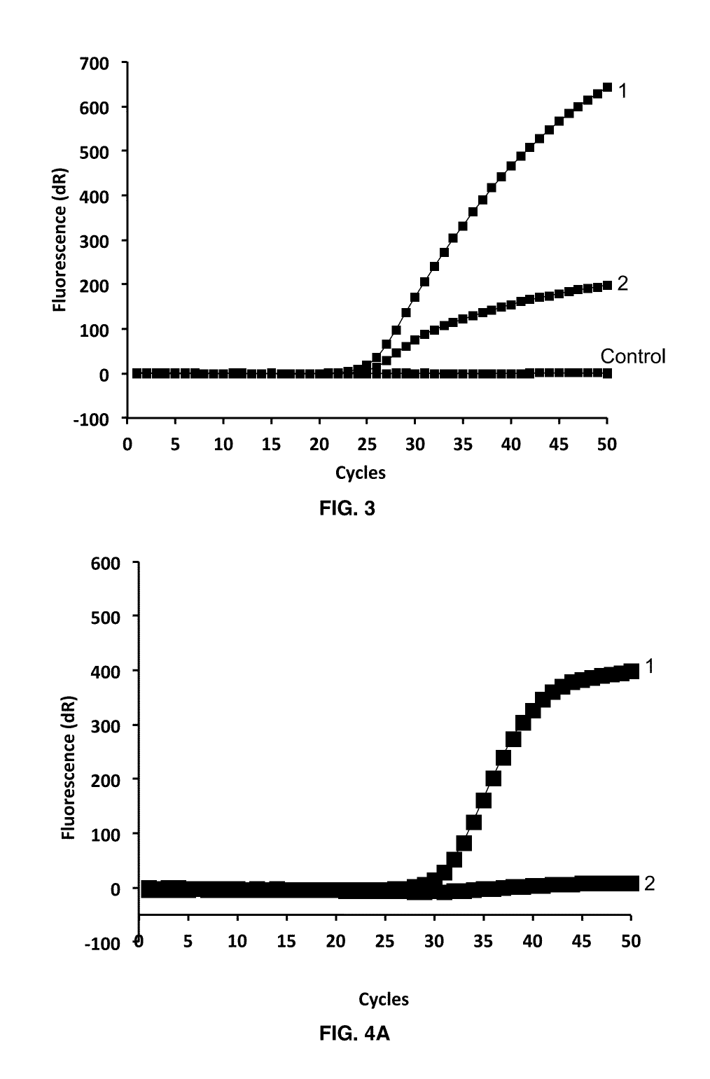

Cycling conditions: Real-time PCR was performed as described in Example 1, except that 50 cycles of 2-step PCR were generally performed. The qPCR results are as expected and are shown and discussed in FIG. 3.

EXAMPLE 4

### Real-time PCR Using iDDS Probes for the Detection of the L858R Point Mutation in the Human Epidermal Growth Factor Receptor (EGFR) Gene

Each tube contained an ULTRAMER® oligonucleotide template comprising the targeted EGFR gene segment. The probes, antiprobes, and primers were fabricated with the following sequences and labeling, and were used at the indicated final concentrations:

EGFR 858 probe 1:
(SEQ ID NO: 19)
Q670-AGCAGTTTGGGCAGCCCAA-BHQ-2
at 250 nM,
EGFR 858 antiprobe 1:
(SEQ ID NO: 20)
TTGGGCTGGCCTAACTGCT-BHQ-2
at 500 nM,
EGFR 858 probe 2:
(SEQ ID NO: 21)
FAM-AGCAGTTTGGGCCGCCC-3IABkFq
at 200 nM,
EGFR 858 antiprobe 2:
(SEQ ID NO: 22)
GGGCGGGCCAAACGGCT-BHQ-1
at 400 nM,
EGFR 858 forward primer:
(SEQ ID NO: 23)
GAAAACACCGCAGCATGTC
at 200 nM,
and
EGFR 858 reverse primer:
(SEQ ID NO: 24)
CTGCATGGTATTCTTTCTCTTCC
at 200 nM.

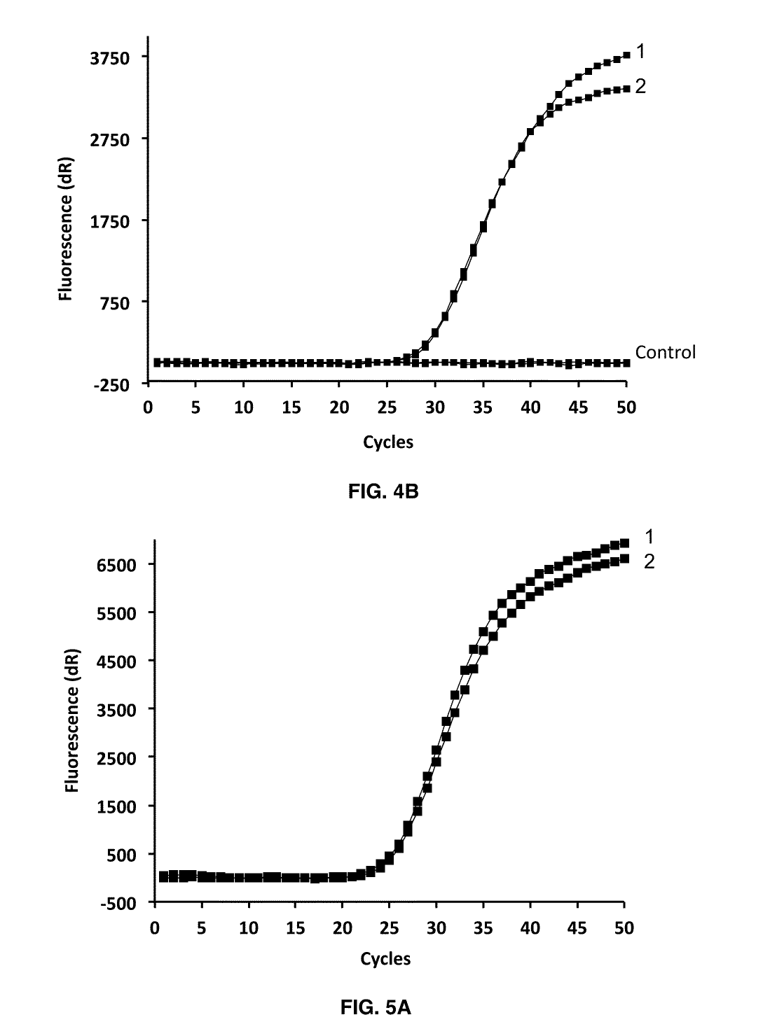

Cycling conditions: Real-time PCR was performed as described in Example 1 except that 50 cycles of 2-step PCR were performed, using an annealing/extension step at 58° C. for 1 min. The results are as expected and examples of the qPCR results are shown and discussed in FIGS. 4A and 4B.

### EXAMPLE 5

### Real-time PCR Using iDDS Probes for the Detection of a Deletion in Exon 19 (D19) of the Human Epidermal Growth Factor Receptor (EGFR) Gene

Each tube contained an ULTRAMER® oligonucleotide template comprising the targeted EGFR gene segment. The probes, antiprobes, and primers were fabricated with the following sequences and labeling, and were used at the indicated final concentrations:

D19 probe 1:
(SEQ ID NO: 25)
Q670-CTCTGGATCCCAGAAGGTGAGA-BHQ-2
at 200 nM,
D19 antiprobe 1:
(SEQ ID NO: 26)
TCTCTCCTTCTGGGATCCAGAG-BHQ-2
at 400 nM,
D19 probe 2:
(SEQ ID NO: 27)
FAM-CAAGGAATTAAGAGAAGCAACATC
at 200 nM,
D19 antiprobe 2:
(SEQ ID NO: 28)
GATGTTGCCTCTCTTAATTCCTTG-BHQ-1
at 400 nM,
D19 forward primer:
(SEQ ID NO: 29)
GGTAACATCCACCCAGATCA
at 200 nM,
and
D19 reverse primer:
(SEQ ID NO: 30)
CATCGAGGATTTCCTTGTTG
at 200 nM.

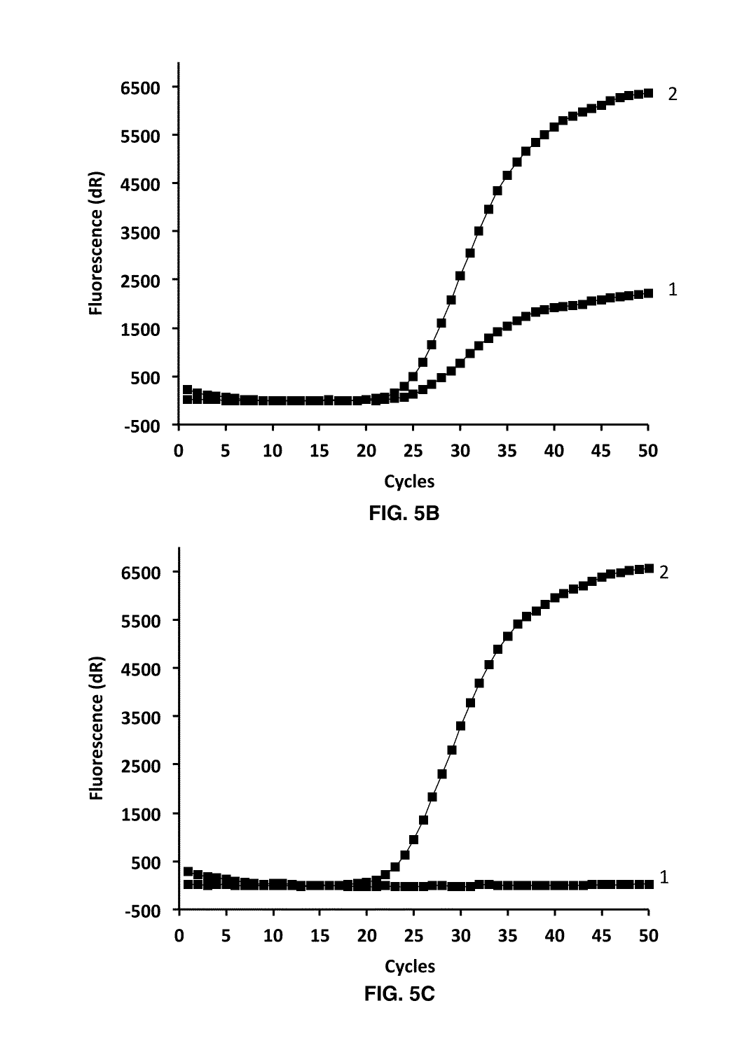

Cycling conditions: Real-time PCR was performed as described in Example 1 except that 50 cycles of 2-step PCR were performed, using an annealing/extension step at 54° C. for 1 min. The results are as expected from qPCR runs with WT and exon 19 deletion mutant templates and are shown and discussed in FIGS. 5A-5C.

### EXAMPLE 6

### Real-time PCR Using iDDS Probes for the Detection of the 5′ Untranslated Region of Echovirus (EV) and Parechovirus (PV)

The templates used were ULTRAMER® oligonucleotides comprising the targeted gene segment, or EV cDNA. The probes, antiprobes, and primers were fabricated with the following sequences and labeling, and were used at the indicated final concentrations:

EV probe 1:
(SEQ ID NO: 31)
Q670-CGGAACCGACTACTTTGGGTG-BHQ-2
at 200 nM,
EV antiprobe 1:
(SEQ ID NO: 32)
CACCCAATGTAGTCGGTTCCG-BHQ-2
at 400 nM,
EV probe 2:
(SEQ ID NO: 33)
FAM-AAACACGGACACCCAAAGTAGTCG-IABkFQ
at 200 nM,
EV antiprobe 2:
(SEQ ID NO: 34)
CGACTACTTTGGTTGTCCGTGTTT-IABkFQ
at 400 nM,
EV forward primer:
(SEQ ID NO: 35)
CCCCTGAATGCGGMTAAT
at 200 nM,
EV reverse primer:
(SEQ ID NO: 36)
RATTGTCACCATAAGCAGCCA
at 200 nM,
PV probe 1:
(SEQ ID NO: 37)
CFO560-CACTATGGATCTGATCTGGGGC-BHQ-1
at 200 nM,
PV antiprobe 1:
(SEQ ID NO: 38)
GCCCCAGATCAGATCCTTAGTG-IABkFQ
at 400 nM,
PV probe 2:
(SEQ ID NO: 39)
CFR610-CGGGTACCTTCTGGGCATC-BHQ-2
at 200 nM,
PV antiprobe 2:
(SEQ ID NO: 40)
GATGCCCTGAAGGTACCCG-IABkRq
at 400 nM,
PV forward primer:
(SEQ ID NO: 41)
GGCCAAAAGCCAAGGTTTA
at 200 nM,
and
PV reverse primer:
(SEQ ID NO: 42)
TTGGCCCACTAGACGTTTTT
at 200 nM.

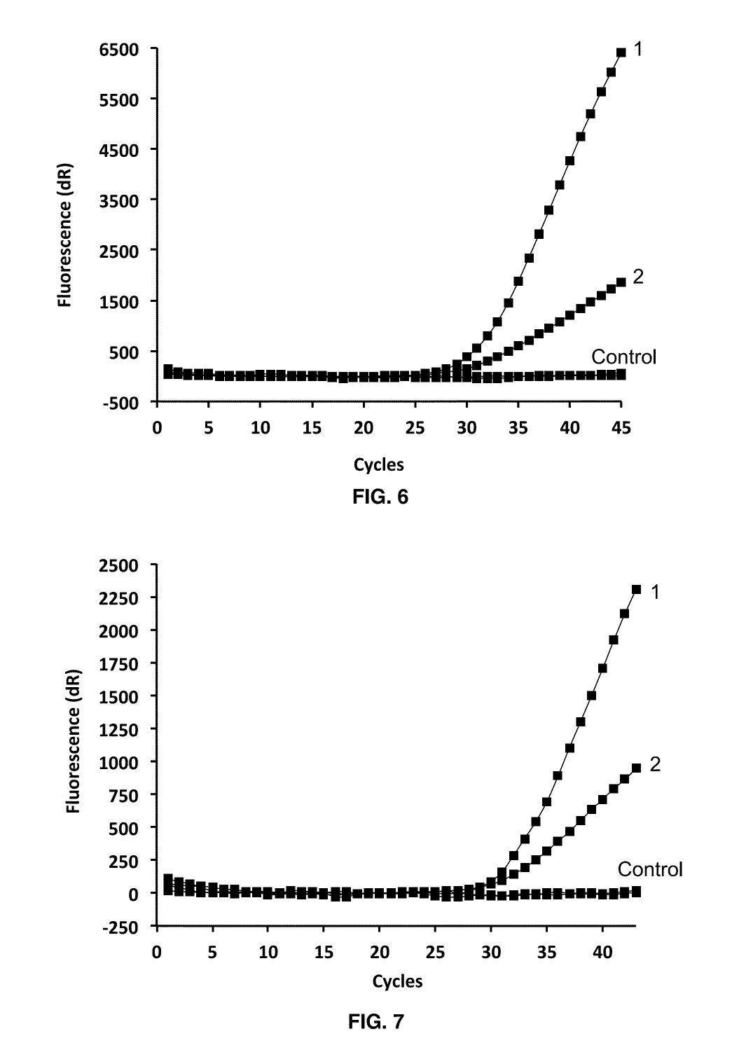

Cycling conditions: Real-time PCR was performed as described in Example 1 except that 40 cycles of 2-step PCR were performed, using an annealing/extension step at 64° C. for 1 min. The results are as expected and examples of qPCR results with the EV and PV probes are shown in FIGS. 6 and 7.

### EXAMPLE 7

### Real-time PCR Using iDDS Probes for the Detection of the L1 Gene of Human Papillomavirus 16 (HPV-16)

Each tube contained an ULTRAMER® oligonucleotide template comprising the targeted HPV16 gene segment. The probes, antiprobes, and primers were fabricated with the following sequences and labeling, and were used at the indicated final concentrations:

probe 1:
(SEQ ID NO: 43)
FAM-CGAGCACAGGGCCACAAT
at 200 nM,
antiprobe 1:
(SEQ ID NO: 44)
ATTGTGGTCCTGTGCTCG-BHQ-1
at 400 nM,
probe 2:
(SEQ ID NO: 45)
CFR610-GGTTACCCCAACAAATGCCA
at 200 nM,
antiprobe 2:
(SEQ ID NO: 46)
TGGCTTTTGTTGGGGTAACC-BHQ-2
at 400 nM,
HPV16 forward primer:
(SEQ ID NO: 47)
GGTTACCTCTGATGCCCAAATA
at 200 nM,
and
HPV16 reverse primer:
(SEQ ID NO: 48)
TTTCTGAAGTAGATATGGCAGCAC
at 200 nM.

Cycling conditions: Real-time PCR is performed as described in Example 1, except that an annealing/extension step at 58° C. for 1 min was used during 50 cycles of 2-step PCR.

### EXAMPLE 8

### Real-time PCR Using iDDS Probes for the Detection of the L1 Gene of Human Papillomavirus 18 (HPV-18)

Each tube contained an ULTRAMER® oligonucleotide template comprising the targeted HPV18 gene segment. The probes, antiprobes, and primers were fabricated with the following sequences and labeling, and were used at the indicated final concentrations:

HPV18 probe 1:
(SEQ ID NO: 49)
FAM-GGTTACATAAGGCACAGGGTCATAAC
at 200 nM,
HPV18 antiprobe 1:
(SEQ ID NO: 50)
GTTATGACCCTGTCCCTTATGTAACC-BHQ-1
at 400 nM,
HPV18 probe 2:
(SEQ ID NO: 51)
CFR610-TTGATTATGCCAGCAAACACCA
at 200 nM,
HPV18 antiprobe 2:
(SEQ ID NO: 52)
TGGAGTTTGCTGGCATAATCAA-BHQ-2
at 400 nM,
HPV18 forward primer:
(SEQ ID NO: 53)
TGTTACCTCTGACTCCCAGTTG
at 200 nM,
and
HPV18 reverse primer:
(SEQ ID NO: 54)
TTGCCCAGGTACAGGAGACT
at 200 nM.

Cycling conditions: Real-time PCR was performed as described in Example 1, except that an annealing/extension step at 58° C. for 1 min was used during 50 cycles of 2-step PCR.

### EXAMPLE 9

### Real-time PCR Using iDDS Probes for the Detection of Seasonal (SEAS) or 2009-pandemic (PDM) H1N1 Influenza A, H3N2 Influenza A (H3N2), or Influenza B (FLU B)

iDDS probes were targeted to signature sites in the hemagglutinin (HA) segment of influenza A genomes, or the nonstructural protein 1 (NS1) of influenza B genomes. Each tube contained an ULTRAMER® oligonucleotide template comprising the targeted influenza A or B gene segments. The probes, antiprobes, and primers were fabricated with the following sequences and labeling, and were used at the indicated final concentrations:

SEAS probe:
(SEQ ID NO: 55)
FAM-TTGGGTAACTGCAGCGTTGC
at 200 nM,
SEAS antiprobe:
(SEQ ID NO: 56)
GCAACGCAGCAGTTACCCAA-BHQ1
at 400 nM,
SEAS forward primer:
(SEQ ID NO: 57)
GCTACCATGCTAACAACTCGAC
at 200 nM,
SEAS reverse primer:
(SEQ ID NO: 58)
TCGCATTCTGGGTTTCC
at 200 nM,
PDM probe:
(SEQ ID NO: 59)
CFO560-TCCGATCACAATTGGAAAATGTC
at 200 nM,
PDM antiprobe:
(SEQ ID NO: 60)
GACTTTTTCCAATTGTGATCGGA-BHQ-2
at 400 nM,
PDM forward primer:
(SEQ ID NO: 61)
ATAAACACCAGCCTCCCATT
at 200 nM,
PDM reverse primer:
(SEQ ID NO: 62)
GTGGCCAGTCTCAATTTTGT
at 200 nM,
H3N2 probe:
(SEQ ID NO: 63)
CFR610-TATGATGTGCCGGATTATGCCT
at 200 nM,
H3N2 antiprobe:
(SEQ ID NO: 64)
AGGCATAATCCGGCTCATCATA-BHQ-2
at 400 nM,
H3N2 forward primer:
(SEQ ID NO: 65)
CCTTTTTGTTGAACGAAGCA
at 200 nM,
H3N2 reverse primer:
(SEQ ID NO: 66)
TCATTGTTAAACTCCAGTGTGC
at 200 nM,
FLU B probe:
(SEQ ID NO: 67)
Q670-CCGTGACCAGTCTAATTGTCTCC
at 200 nM,
FLU B antiprobe:
(SEQ ID NO: 68)
GGAGACATTTAGACTGGTCACGG-IABkRq
at 400 nM,
FLU B forward primer:
(SEQ ID NO: 69)
GTCAAGAGCACCGATTATCAC
at 200 nM,
and
FLU B reverse primer:
(SEQ ID NO: 70)
CAACCATGTCAGCTATTATGGAG
at 200 nM.

All four probes and antiprobes were used together in the same tube. Each tube contained a different influenza A or B template. Cycling conditions: Real-time PCR was performed as described in Example 1, except that an annealing/extension step at 58° C. for 1 min was used during 50 cycles of 2-step PCR.

### EXAMPLE 10

### Real-time PCR Using iDDS Probes for the Detection T-cell Receptor Excision Circles (TRECs) Found in Patients with Severe Combined Immunodeficiency

Each tube contained either an ULTRAMER® oligonucleotide template comprising the targeted TREC gene segment, or DNA extracted from a clinical sample. The probes, antiprobes, and primers were fabricated with the following sequences and labeling, and were used at the indicated final concentrations:

TREC probe 1:
(SEQ ID NO: 71)
FAM-TTGTAAAGGTGCCCACTCCTGT
at 200 nM,
TREC antiprobe 1:
(SEQ ID NO: 72)
ACAGGAGTGGGCTCCTTTACAA-BHQ-1
at 400 nM,
TREC probe 2:
(SEQ ID NO: 73)
Cy5-TGCAGGTGCCTATGCATCAC
at 200 nM,
TREC antiprobe 2:
(SEQ ID NO: 74)
GTGATGCTTAGGCACCTGCA-IABkRq
at 400 nM,
TREC forward primer:
(SEQ ID NO: 75)
TTCAACCATGCTGACACCTC
at 200 nM,
and
TREC reverse primer:
(SEQ ID NO: 76)
CAGCTGCAGGGTTTAGGC
at 200 nM.

Both probes and antiprobes are used together in the same tube.

Cycling conditions: Real-time PCR was performed as described in Example 1, except that an annealing/extension step at 58° C. for 1 min was used during 50 cycles of 2-step PCR.

### EXAMPLE 11

### Real-time PCR Using Half-Universal Probes for the Detection of Human Immunodeficiency Virus (HIV) Gag and Pol Genes

Each tube contained an ULTRAMER® oligonucleotide template comprising the targeted HIV gene segment. The probes, antiprobe, and primers were fabricated with the following sequences and labeling, and were used at the indicated final concentrations:

Gag probe:
(SEQ ID NO: 77)
FAM-CTACGTAGACTAGACGTTCACCTATCCCAGTGGGAGAGAT
at 80 nM,
Half-Universal antiprobe:
(SEQ ID NO: 78)
GAACGTCTAGTCTACGTAG-BHQ-1
at 320 nM,
Gag primer:
(SEQ ID NO: 79)
ATGCTGACAGGGCTATACATTCTT
at 80 nM,
Pol probe:
(SEQ ID NO: 80)
CY3-CTACGTAGACTAGACGTTCCAGAGCAGACCAGAGCCA
at 80 nM,
and
Pol primer:
(SEQ ID NO: 81)
AGGGGTCGTTGCCAAAGAGTGAT
at 80 nM.

Both Half-Universal probes and antiprobes were used together in the same tube.

Cycling conditions: Real-time PCR was performed as described in Example 1, except that 50 cycles of 3-step PCR were performed, using a denaturing step at 95° C. for 15 seconds, an annealing step at 58° C. for 30 seconds, and an extension step at 72° C. for 30 seconds.

### EXAMPLE 12

### Real-time PCR Using iDDS Probes for the Detection of Point Mutants of Human Isocitrate Dehydrogenase Genes IDH1 or IDH2

Each tube contained an ULTRAMER® oligonucleotide template comprising the targeted IDH1 or IDH2 gene segment. The probes, antiprobe, and primers were fabricated with the following sequences and labeling, and were used at the indicated final concentrations:

Mutant IDH1 iDDS probe 1:
(SEQ ID NO: 82)
FAM-CATAGGTCATGATGCTTATGGGGA-BHQ1
at 200 nM,
Mutant IDH1 iDDS antiprobe 1:
(SEQ ID NO: 83)
TCCGCATAAGCATGATGACCTATG-BHQ1
at 200 nM,
Mutant IDH1 iDDS probe 2:
(SEQ ID NO: 84)
CFR610-CCATAAGCATGACAAGCTATGATG-BHQ2
at 150 nM,
Mutant IDH1 iDDS antiprobe 2:
(SEQ ID NO: 85)
CATCATAGGTTGTCTTGCTTATGG-BHQ2
at 400 nM,
IDH1 forward primer:
(SEQ ID NO: 86)
GGCTTGTGAGTGGATGGGTA
at 200 nM,
IDH1 reverse primer:
(SEQ ID NO: 87)
AAAACATGCAAAATCACATTAT
at 200 nM,
Mutant IDH2 iDDS probe 1:
(SEQ ID NO: 88)
FAM-CCATTGGCAAGGACGCCC-BHQ1
at 150 nM,
Mutant IDH2 iDDS antiprobe 1:
(SEQ ID NO: 89)
GGGCGTGCTTGCCTATGG-BHQ1
at 400 nM,
Mutant IDH2 iDDS probe 2:
(SEQ ID NO: 90)
CFR610-CCATTGGCATGGACGCCC-BHQ2
at 150 nM,
Mutant IDH2 iDDS antiprobe 2:
(SEQ ID NO: 91)
GGGCGTGCATGCCTATGG-BHQ2
at 400 nM,
IDH2 forward primer:
(SEQ ID NO: 92)
CACTATTATCTCTGTCCTCA
at 200 nM,
and
IDH2 reverse primer:
(SEQ ID NO: 93)
AGACAAGAGGATGGCTA
at 200 nM.

Cycling conditions: Real-time PCR was performed as described in Example 1, except that the annealing/extension step was set at variable conditions ranging from 50-62° C. for 1 min, depending upon which combination of probes were run together in the same tube.

It should be emphasized that the embodiments of the present disclosure, particularly, any “preferred” embodiments, are merely possible examples of the implementations, merely set forth for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure, and the present disclosure and protected by the following claims.

What is claimed is:

1. A multi-probe system to improve signaling and/or reliability in detection of a genus, a species, a gene or other related nucleic acid targets during real time PCR or other amplification methods, the system comprising:
(i) two or more probes complementary to two or more related signature sequences in a sample, said probes having identical labels as signal amplifiers, or having different labels for confirmation of detection, or having both identical and different labels; wherein the probes comprise one or more probe:antiprobe systems selected from the group consisting of: iDDS probes, iDDS-2Q probes, MacMan probes, Flip probes, Universal probes, Half-Universal probes, ZIPR probes, and G-Force probes; and
(ii) two flanking primer components comprising two primers, one primer or one primer-probe, or two primer-probes.

2. The multi-probe system of claim 1, wherein the probes or antiprobes further comprise one or more synthetic nucleotides.

3. The multi-probe system of claim 1, wherein the system further comprises one or more alternate probes selected from the group consisting of TaqMan probes, Molecular Beacon probes, Scorpion probes, LUX probes, Sunrise probes and Dual Hybridization probes; or wherein one probe comprising the system is replaced with an alternate probe.

4. The multi-probe system of claim 1, wherein the two or more related signature sequences are selected from the group of genes or species consisting of a vanA gene of Vancomycin resistant Enterococcus (VRE), a vanB gene of VRE, a Trichomonas vaginalis-specific repeated sequence (GenBank: L23861.1), an EGFR 858 mutant site, an EGFR exon 19 deletion site, a 5′UTR region of Enterovirus or Parechovirus or a combination thereof, an L1 gene of human papillomavirus 16 (HPV-16), an L1 gene of human papillomavirus 18 (HPV-18), an HA gene of influenza A or the NS1 gene of influenza B or a combination thereof, a human T-cell receptor excision circle (TREC) DNA, Gag and Pol genes of HIV-1, and an exon 4 mutant sites of the human isocitrate dehydrogenase genes IDH1 and IDH2.

5. The multi-probe system of claim 4, wherein the related signature sequence is the vanA gene nucleic acid sequence with GenBank Accession No. M97297.1, the system comprising a pair of flanking primers specific to the vanA coding domain sequence (CDS) in the region from about nucleotide 7316 to about nucleotide 7381 and in the region from about nucleotide 7438 to about nucleotide 7488, and/or two iDDS probe:antiprobe systems specific to the vanA CDS in the region from about nucleotide 7368 to about nucleotide 7413 and in the region from about nucleotide 7407 to about 7451, or a combination thereof.

6. The multi-probe system of claim 5,
wherein the primers comprise:
a forward primer sequence shown in SEQ ID NO: 5, and
a reverse primer sequence shown in SEQ ID NO: 6;
wherein the probe:antiprobe system comprises:
a first iDDS probe sequence shown in SEQ ID NO: 1,
a first iDDS antiprobe sequence shown in SEQ ID NO: 2,
a second iDDS probe sequence shown in SEQ ID NO: 3, and
a second iDDS antiprobe sequence shown in SEQ ID NO: 4; and
wherein the probes are labeled with a fluorophore or with a fluorophore and a quencher, and the antiprobes are labeled with a quencher.

7. The multi-probe system of claim 4, wherein the related signature sequence is the vanB gene nucleic acid sequence with GenBank Accession No. CP003351.1, the system comprising a pair of flanking primers specific to the vanB coding domain sequence (CDS) in the region from about nucleotide 2839810 to about nucleotide 2840049 and in the region from about nucleotide 2839810 to about nucleotide 2840049, and/or two iDDS probe:antiprobe systems specific to the vanB CDS in the region from about nucleotide 2839878 to about nucleotide 2839958 and in the region from about nucleotide 2839936 to about 2839995, or a combination thereof.

8. The multi-probe system of claim 7,
wherein the primers comprise:
a forward primer sequence shown in SEQ ID NO: 11, and
a reverse primer sequence shown in SEQ ID NO: 12;
wherein the probe:antiprobe system comprises:
a first iDDS probe sequence shown in SEQ ID NO: 7,
a first iDDS antiprobe sequence shown in SEQ ID NO: 8,
a second iDDS probe sequence shown in SEQ ID NO: 9, and
a second iDDS antiprobe sequence shown in SEQ ID NO: 10; and
wherein the probes are labeled with a fluorophore or with a fluorophore and a quencher, and the antiprobes are labeled with a quencher.

9. The multi-probe system of claim 4, wherein the related signature sequence is the Trichomonas vaginalis (T. vag.) repetitive nucleic acid sequence with GenBank Accession No. L23861.1, the system comprising a pair of flanking primers specific to the T. vag. sequence in the region from about nucleotide 580 to about nucleotide 639 and in the region from about nucleotide 678 to about nucleotide 737, and/or two iDDS probe:antiprobe systems specific to the T. vag. sequence in the region from about nucleotide 623 to about nucleotide 659 and in the region from about nucleotide 645 to about 686, or a combination thereof.

10. The multi-probe system of claim 9,
wherein the primers comprise:
a forward primer sequence shown in SEQ ID NO: 17, and
a reverse primer sequence shown in SEQ ID NO: 18;
wherein the probe:antiprobe system comprises:
a first iDDS probe sequence shown in SEQ ID NO: 13,
a first iDDS antiprobe sequence shown in SEQ ID NO: 14,
a second iDDS probe sequence shown in SEQ ID NO: 16, and
a second iDDS antiprobe sequence shown in SEQ ID NO: 16; and
wherein the probes are labeled with a fluorophore or with a fluorophore and a quencher, and the antiprobes are labeled with a quencher.

11. The multi-probe system of claim 4, wherein the related signature sequence is the wild type (WT) EGFR nucleic acid sequence or the L858R mutant EGFR nucleic acid sequence with GenBank Accession No. NW_{—}004078029.1 or both, the system comprising a pair of flanking primers specific to the EGFR DNA sequence in the region from about nucleotide 55207745 to about nucleotide 55207805 and in the region from about nucleotide 55207824 to about nucleotide 55207884, and/or two iDDS probe:antiprobe systems specific to the WT or mutant EGFR sequence in the region from about nucleotide 55207792 to about 55207832, or a combination thereof.

12. The multi-probe system of claim 11,
wherein the primers comprise:
a forward primer sequence shown in SEQ ID NO: 23, and
a reverse primer sequence shown in SEQ ID NO: 24;
wherein the probe:antiprobe system comprises:
a WT iDDS probe sequence shown in SEQ ID NO: 19,
a WT iDDS antiprobe sequence shown in SEQ ID NO: 20,
a mutant iDDS probe sequence shown in SEQ ID NO: 21, and
a mutant iDDS antiprobe sequence shown in SEQ ID NO: 22; and
wherein the probes are labeled with a fluorophore or with a fluorophore and a quencher, and the antiprobes are labeled with a quencher.

13. The multi-probe system of claim 4, wherein the related signature sequences are a variable exon 19 deletion mutant sequence and an adjacent reference sequence in the human EGFR gene with GenBank Accession number NW_{—}004078029.1), said system comprising a pair of flanking primers specific to the EGFR sequence in the region from about nucleotide 55242321 to about nucleotide 55242414 and about 55242489-55242537, and/or two iDDS probe:antiprobe pairs specific to an EGFR WT or deletion mutant target sequence in the region from about nucleotide 55242412 to about nucleotide 55242444 and from about nucleotide 55242458 to about nucleotide 55242489, respectively, or a combination thereof.

14. The multi-probe system of claim 13,
wherein the primers comprise:
a forward primer sequence shown in SEQ ID NO: 29, and
a reverse primer sequence shown in SEQ ID NO: 30;
wherein the probe:antiprobe system comprises:
a first iDDS probe sequence shown in SEQ ID NO: 25,
a first iDDS antiprobe sequence shown in SEQ ID NO: 26,
a second iDDS probe sequence shown in SEQ ID NO: 27, and
a second iDDS antiprobe sequence shown in SEQ ID NO: 28; and
wherein the probes are labeled with a fluorophore or a fluorophore and a quencher, and the antiprobes are labeled with a quencher.

15. The multi-probe system of claim 4, wherein the related signature sequence is a 5′-untranslated region of enterovirus (EV) sequence with GenBank Accession No. HQ728259.1 or a parechovirus (PV) nucleic acid sequence with GenBank Accession No. EF051629.2, said system comprising a pair of flanking primers specific to EV DNA in the region from about nucleotide 446 to about nucleotide 487 and from about nucleotide 583 to about nucleotide 642, a pair of flanking primers specific to PV DNA in the region from about nucleotide 420 to about nucleotide 461 and from about nucleotide 641 to about nucleotide 680, and/or two iDDS probe:antiprobe systems specific to the EV sequence in the region from about nucleotide 527 to about nucleotide 603, and/or two iDDS probe:antiprobe systems specific to the PV sequence in the region from about nucleotide 530 to about nucleotide 590 and from about nucleotide 556 to about nucleotide 616, or combinations thereof.

16. The multi-probe system of claim 15,
wherein the primers comprise:
a forward EV primer sequence shown in SEQ ID NO: 35,
a reverse EV primer sequence shown in SEQ ID NO: 36,
a PV forward primer sequence shown in SEQ ID NO: 41, and
a PV reverse primer sequence shown in SEQ ID NO: 42;
wherein the probe:antiprobe system comprises:
a first EV iDDS probe sequence shown in SEQ ID NO: 31,
a first EV iDDS antiprobe sequence shown in SEQ ID NO: 32,
a second EV iDDS probe sequence shown in SEQ ID NO: 33,
a second EV iDDS antiprobe sequence shown in SEQ ID NO: 34,
a first PV iDDS probe sequence shown in SEQ ID NO: 37,
a first PV iDDS antiprobe sequence shown in SEQ ID NO: 38,
a second PV iDDS probe sequence shown in SEQ ID NO: 39, and
a second PV iDDS antiprobe sequence shown in SEQ ID NO: 40; and
wherein the probes are labeled with a fluorophore or a fluorophore and a quencher, and the antiprobes are labeled with a quencher.

17. The multi-probe system of claim 4, wherein the related signature sequence is a human papillomavirus 16 (HPV16) L1 gene nucleic acid sequence with GenBank Accession No. EU430688.1), comprising a pair of flanking primers specific to the L1 CDS in the nucleotide region from about nucleotide 938 to about nucleotide 1020 and from about nucleotide 1077 to about nucleotide 1155, and/or two iDDS probe:antiprobe systems specific to the L1 CDS in the region from about nucleotide 1020 to about nucleotide 1050 and from about nucleotide 1032 to about nucleotide 1063, or a combination thereof.

18. The multi-probe system of claim 17,
wherein the primers comprise:
a forward primer sequence shown in SEQ ID NO: 47, and
a reverse primer sequence shown in SEQ ID NO: 48;
wherein the probe:antiprobe system comprises:
a first iDDS probe sequence shown in SEQ ID NO: 43,
a first iDDS antiprobe sequence shown in SEQ ID NO: 44,
a second iDDS probe sequence shown in SEQ ID NO: 45, and
a second iDDS antiprobe sequence shown in SEQ ID NO: 46; and
wherein the probes are labeled with a fluorophore or a fluorophore and a quencher, and the antiprobes are labeled with a quencher.

19. The multi-probe system of claim 4, wherein the related signature sequence is a human papillomavirus 18 (HPV18) L1 gene nucleic acid sequence with GenBank Accession No. EU834744.1, comprising a pair of flanking primers specific to the HPV18 CDS in the region from about nucleotide 1032 to about nucleotide 1103 and from about nucleotide 1196 to about nucleotide 1287, and/or two iDDS probe:antiprobe systems specific to the HPV18 CDS in the region from about nucleotide 1098 to about nucleotide 1160 and from about nucleotide 1125 to about nucleotide 1185, or a combination thereof.

20. The multi-probe system of claim 19,
wherein the primers comprise:
a forward primer sequence shown in SEQ ID NO: 53, and
a reverse primer sequence shown in SEQ ID NO: 54;
wherein the probe:antiprobe system comprises:
a first iDDS probe sequence shown in SEQ ID NO: 49,
a first iDDS antiprobe sequence shown in SEQ ID NO: 50,
a second iDDS probe sequence shown in SEQ ID NO: 51, and
a second iDDS antiprobe sequence shown in SEQ ID NO: 52; and
wherein the probes are labeled with a fluorophore or a fluorophore and a quencher, and the antiprobes are labeled with a quencher.

21. The multi-probe system of claim 4, wherein the related signature sequence is an influenza A or B nucleic acid sequence, said system comprising four sets of flanking primers and associated iDDS probe:antiprobes, comprising:
(i) a first set of a pair of flanking primers specific to the hemagglutinin (HA) CDS of an H1N1 seasonal influenza A with GenBank Accession No. CY069381.1 in the region from about nucleotide 25 to about nucleotide 95 and from about nucleotide 225 to about nucleotide 295, and an iDDS probe:antiprobe system specific to the HA CDS in the region from about nucleotide 185 to about nucleotide 245;
(ii) a second set of a pair of flanking primers specific to the HA CDS of a 2009 H1N1 pandemic influenza A with GenBank Accession No. FJ969540.1 in the region from about nucleotide 878 to about nucleotide 938 and from about nucleotide 971 to about nucleotide 1061, and an iDDS probe:antiprobe system specific to the HA CDS in the region from about nucleotide 920 to about nucleotide 980;
(iii) a third set of a pair of flanking primers specific to the HA CDS of an H3N2 influenza A with GenBank Accession No. CY120904 in the region from about nucleotide 285 to about nucleotide 345 and from about nucleotide 370 to about nucleotide 430, and an iDDS probe:antiprobe system specific to the HA CDS in the region from about nucleotide 329 to about nucleotide 389; and
(iv) a fourth set of a pair of flanking primers specific to the NS1 CDS of an influenza B with GenBank Accession No. JQ034241.1 in the region from about nucleotide 770 to about nucleotide 830 and from about nucleotide 892 to about nucleotide 952, and an iDDS probe:antiprobe system specific to the HA CDS in the nucleotide region from about nucleotide 831 to about nucleotide 891.

22. The multi-probe system of claim 21,
wherein the primers comprise:
the first set forward primer with a sequence shown in SEQ ID NO: 57 and the first set reverse primer with a sequence shown in SEQ ID NO: 58,
the second set forward primer with a sequence shown in SEQ ID NO: 61 and the second set reverse primer with a sequence shown in SEQ ID NO: 62,
the third set forward primer with a sequence shown in SEQ ID NO: 65 and the third set reverse primer with a sequence shown in SEQ ID NO: 66, and
the fourth set forward primer with a sequence shown in SEQ ID NO: 69 and the fourth set reverse primer with a sequence shown in SEQ ID NO: 70;
wherein the probe:antiprobe system comprises:
the first set iDDS probe with a sequence shown in SEQ ID NO: 55 and the first set iDDS antiprobe with a sequence shown in SEQ ID NO: 56;
the second set iDDS probe with a sequence shown in SEQ ID NO: 59 and the second set iDDS antiprobe with a sequence shown in SEQ ID NO: 60;
the third set iDDS probe with a sequence shown in SEQ ID NO: 63 and the third set iDDS antiprobe with a sequence shown in SEQ ID NO: 64; and
the fourth set iDDS probe with a sequence shown in SEQ ID NO: 67 and the fourth set iDDS antiprobe with a sequence shown in SEQ ID NO: 68; and
wherein the probes are labeled with a fluorophore or a fluorophore and a quencher, and the antiprobes are labeled with a quencher.

23. The multi-probe system of claim 4, wherein the related signature sequence is T-cell receptor excision circle (TREC) nucleic acid sequences derived from excision of Human Chromosome 14 DNA with GenBank Accession No. NW_{—}004078079.1, said system comprising a pair of flanking primers specific to the TREC sequence in the region from about nucleotide 3941495 to about nucleotide 3941555 and from about nucleotide 3853115 to about nucleotide 3853175, and/or two iDDS probe:antiprobe systems specific to the TREC sequence in the region from about nucleotide 3941545 to about nucleotide 3941580 and from about nucleotide 3853091 to about nucleotide 3853120, or a combination thereof.

24. The multi-probe system of claim 23,
wherein the primers comprise:
a forward primer with a sequence shown in SEQ ID NO: 75 and
a reverse primer with a sequence shown in SEQ ID NO: 76;
wherein the probe:antiprobe system comprises:
a first iDDS probe with a sequence shown in SEQ ID NO: 71,
a first iDDS antiprobe with a sequence shown in SEQ ID NO: 72,
a second iDDS probe with a sequence shown in SEQ ID NO: 73, and
a second iDDS antiprobe with a sequence shown in SEQ ID NO: 74; and
wherein the probes are labeled with a fluorophore or a fluorophore and a quencher, and the antiprobes are labeled with a quencher.

25. The multi-probe system of claim 4, wherein the related signature sequences are a human immunodeficiency virus (HIV) Gag nucleic acid sequence with GenBank Accession No. JX244938.1 and a human immunodeficiency virus (HIV) Pol gene nucleic acid sequence with GenBank Accession No. KC462191.1, said system comprising a flanking primer specific to the Gag CDS in the region from about nucleotide 790 to about nucleotide 850, a first Half-Universal probe:antiprobe system specific to the Gag CDS in the region from about nucleotide 729 to about nucleotide 789, a flanking primer specific to the Pol CDS in the region from about nucleotide 1423 to about nucleotide 1483, a second Half-Universal probe:antiprobe system specific to the Pol CDS in the region from about nucleotide 1305 to about nucleotide 1365, or a combination thereof.

26. The multi-probe system of claim 25,
wherein the primers comprise:
the Gag primer with a sequence shown in SEQ ID NO: 79 and
the Pol primer with a sequence shown in SEQ ID NO: 81;
wherein the probe:antiprobe system comprises:
the Gag Half-Universal probe with a sequence shown in SEQ ID NO: 77,
the Pol Half-Universal probe with a sequence shown in SEQ ID NO: 80, and
the Half-Universal antiprobe with a sequence shown in SEQ ID NO: 78; and
wherein the probes are labeled with a fluorophore, and the antiprobes are labeled with a quencher.

27. The multi-probe system of claim 4, wherein the related signature sequences are point mutations in the human IDH1 gene with GenBank Accession No. NT_{—}005403.17 or the IDH2 gene with GenBank Accession No. NC_{—}000015.9, said system comprising a pair of flanking primers specific to IDH1 DNA in the region from about nucleotide 59322435 to about nucleotide 59322519 and from about nucleotide 59322545 to about nucleotide 59322614, and a pair of flanking primers specific to IDH2 DNA in the region from about nucleotide 90631711 to about nucleotide 90631770 and from about nucleotide 90631981 to about 90632040, and/or two iDDS probe:antiprobe systems specific to mutations in the IDH1 sequence in the region from about nucleotide 59322510 to about nucleotide 59322550 or in the IDH2 sequence from about nucleotide 90631818 to about nucleotide 90631857, or combinations thereof.

28. The multi-probe system of claim 27,
wherein the primers comprise:
a forward IDH1 primer sequence shown in SEQ ID NO: 86,
a reverse IDH1 primer sequence shown in SEQ ID NO: 87;
a forward IDH2 primer sequence shown in SEQ ID NO: 92, and
a reverse IDH2 primer sequence shown in SEQ ID NO: 93;
wherein the probe:antiprobe system comprises:
a first mutant IDH1 iDDS probe sequence shown in SEQ ID NO: 82 or a sequence with one or more synthetic nucleotides,
a first mutant IDH1 iDDS antiprobe sequence shown in SEQ ID NO: 83,
a second mutant IDH1 iDDS probe sequence shown in SEQ ID NO: 84,
a second mutant IDH1 iDDS antiprobe sequence shown in SEQ ID NO: 85;
a first mutant IDH2 iDDS probe sequence shown in SEQ ID NO: 88,
a first mutant IDH2 iDDS antiprobe sequence shown in SEQ ID NO: 89,
a second mutant IDH2 iDDS probe sequence shown in SEQ ID NO: 90, and
a second mutant IDH2 iDDS antiprobe sequence shown in SEQ ID NO: 91; and
wherein the probes are labeled with a fluorophore or a fluorophore and a quencher and the antiprobes are labeled with a quencher.

29. The multi-probe system of claim 4, wherein one or more probes are configured for sequential or separate use in target detection.

30. The multi-probe system of claim 1, wherein at least one related signature sequence is selected from the group of genes or species consisting of:
(1) exon 2, codon 12 or codon 13 or both mutant sites of a KRAS gene with GenBank Accession No. NM_{—}004985,
(2) a codon 600 mutant site of the BRAF gene with GenBank Accession No. NM_{—}004333.4,
(3) a Pseudomonas aeruginosa algD gene with GenBank Accession No. CP000438,
(4) a Burkholderia cepacia complex recA gene with GenBank Accession No. NC 018513.1,
(5) a Stenotrophomonas maltophilia 23S gene with GenBank Accession No. NC_{—}015947,
(6) a Achromobacter species OXA-114-like gene with GenBank Accession No. EU188842.1,
(7) a Staphylococcus aureus femA gene with GenBank Accession No. CP003194,
(8) a Staphylococcus aureus mecA gene with GenBank Accession No. AB033763.2,
(9) a Mycobacterium avium complex intergenic spacer gene with GenBank Accession No. L07855.2, or the Mycobacterium chelonae with GenBank Accession No. DQ866771.1 or both, and
(10) a Mycobacterium abscessus (GenBank: HG313848.1) (MCAG) intergenic spacer gene.

31. The multi-probe system of claim 30, wherein the related signature sequences are a reference sequence and an adjacent mutant region in exon 2, codon 12 or 13 of the human KRAS gene, said system comprising a flanking primer specific to the KRAS sequence in the region from about nucleotide 144 to about nucleotide 204, a ZIPR reference probe specific to the WT KRAS sequence in the region from about nucleotide 231 to about nucleotide 291, a WT iDDS probe specific to a presumptive mutant sequence in codon 12 or codon 13 in the region from about nucleotide 205 to about nucleotide 230, or a combination thereof.

32. The multi-probe system of claim 31,
wherein the primers comprise:
a forward primer sequence shown in SEQ ID NO: 94;
wherein the probe:antiprobe system comprises:
the iDDS probe sequence shown in SEQ ID NO: 95 or a sequence with one or more synthetic nucleotides,
an iDDS antiprobe sequence shown in SEQ ID NO: 96;
the reference ZIPR probe sequence shown in SEQ ID NO: 97, and
a ZIPR antiprobe sequence shown in SEQ ID NO: 98; and
wherein the iDDS probe is labeled with a fluorophore or a fluorophore and a quencher, the iDDS antiprobe is labeled with a quencher, the ZIPR probe is labeled with a fluorophore, and the ZIPR antiprobe is labeled with a quencher.

33. The multi-probe system of claim 30, wherein the related signature sequence is a mutant site in codon 600 of the human BRAF gene or a WT reference sequence of BRAF or both, said system comprising a pair of flanking primers specific to the BRAF sequence in the region from about nucleotide 1785 to about nucleotide 1844 and from about nucleotide 1876 to about nucleotide 1935, a V600E mutant iDDS probe and a WT iDDS probe in the region from about nucleotide 1845 to about nucleotide 1875.

34. The multi-probe system of claim 33,
wherein the primers comprise:
the primer sequences shown in SEQ ID NO: 99 and SEQ ID NO: 100;
wherein the probe:antiprobe system comprises:
the mutant iDDS probe sequence shown in SEQ ID NO: 101 or a sequence with one or more synthetic nucleotides, or a combination thereof,
the mutant iDDS antiprobe sequence shown in SEQ ID NO: 102;
the WT iDDS probe with a sequence shown in SEQ ID NO: 103, and
the WT iDDS antiprobe with a sequence shown in SEQ ID NO: 104; and
wherein the probes are labeled with a fluorophore or a fluorophore and a quencher and the antiprobes are labeled with a quencher.

35. The multi-probe system of claim 30, wherein the related signature sequence is from at least one bacterial species commonly associated with cystic fibrosis, said system comprising eight sets of flanking primers or associated iDDS probe:antiprobe systems or both, comprising:
(i) a first set of flanking primers specific to the Pseudomonas aeruginosa algD gene with GenBank Accession No. CP000438.1 in the region from about nucleotide 1595235 to about nucleotide 1595294 and from about nucleotide 1595355 to about nucleotide 1595414, an iDDS probe:antiprobe system specific to the Pseudomonas aeruginosa algD gene in the region from about nucleotide 1595295 to about nucleotide 1595354, or a combination thereof;
(ii) a second set of flanking primers specific to the Burkholderia cepacia complex recA gene with GenBank Accession No. NC_{—}018513.1 in the region from about nucleotide 869329 to about nucleotide 869388 and from about nucleotide 869460 to about nucleotide 869519, an iDDS probe:antiprobe system specific to the Burkholderia cepacia complex recA gene in the region from about nucleotide 869389 to about nucleotide 869448, or a combination thereof;
(iii) a third set of flanking primers specific to the Stenotrophomonas maltophilia 23S gene with GenBank Accession No. NC_{—}015947 in the region from about nucleotide 368121 to about nucleotide 368180 and from about nucleotide 368216 to about nucleotide 368275, an iDDS probe:antiprobe system specific to the Stenotrophomonas maltophilia 23S gene in the region from about nucleotide 368179 to about nucleotide 368215, or a combination thereof;
(iv) a fourth set of flanking primers specific to the Achromobacter species OXA-114-like gene with GenBank Accession No. EU188842.1 in the region from about nucleotide 230 to about nucleotide 289 and from about nucleotide 350 to about nucleotide 409, an iDDS probe:antiprobe system specific to the Achromobacter species OXA-114-like gene in the nucleotide region from about nucleotide 290 to about nucleotide 349, or a combination thereof;
(v) a fifth set of flanking primers specific to the Staphylococcus aureus femA gene with GenBank Accession No. CP003194.1 in the region from about nucleotide 1410168 to about nucleotide 1410227 and from about nucleotide 1410228 to about nucleotide 1410347, an iDDS probe:antiprobe system specific to the Staphylococcus aureus femA gene in the region from about nucleotide 1410228 to about nucleotide 1410287, or a combination thereof;
(vi) a sixth set of flanking primers specific to the Staphylococcus aureus mecA gene with GenBank Accession No. AB033763.2 in the region from about nucleotide 31680 to about nucleotide 31739 and from about nucleotide 31800 to about nucleotide 31859, an iDDS probe:antiprobe system specific to the Staphylococcus aureus mecA gene in the nucleotide region from about nucleotide 31740 to about nucleotide 31799, or a combination thereof;
(vii) a seventh set of flanking primers specific to the Mycobacterium avium complex intergenic spacer gene with GenBank Accession No. L07855.2 in the region from about nucleotide 15 to about nucleotide 74 and from about nucleotide 135 to about nucleotide 194, an iDDS probe:antiprobe system specific to the Mycobacterium avium complex intergenic spacer gene in the region from about nucleotide 75 to about nucleotide 134, or a combination thereof; and
(viii) an eighth set of common flanking primers specific to both the Mycobacterium chelonae intergenic spacer gene with GenBank Accession No. DQ866771.1 in the region from about nucleotide 1461 to about nucleotide 1520 and from about nucleotide 1571 to about nucleotide 1630, the Mycobacterium abscessus intergenic spacer gene with GenBank Accession No. HG313848.1 in the region from about nucleotide 26 to about nucleotide 85 and from about nucleotide 146 to about nucleotide 205, an iDDS probe:antiprobe system specific to the Mycobacterium chelonae intergenic spacer gene in the region from about nucleotide 1521 to about nucleotide 1570, an iDDS probe:antiprobe system specific to the Mycobacterium abscessus intergenic spacer gene in the region from about nucleotide 86 to about nucleotide 145, or combinations thereof.

36. The multi-probe system of claim 35,
wherein the primers comprise:
the first set forward primer sequence shown in SEQ ID NO: 105 and the first set reverse primer sequence shown in SEQ ID NO: 106;
the second set forward primer sequence shown in SEQ ID NO: 109 and the second set reverse primer sequence shown in SEQ ID NO: 110;
the third set forward primer sequence shown in SEQ ID NO: 113 and the third set reverse primer sequence shown in SEQ ID NO: 114;
the fourth set forward primer sequence shown in SEQ ID NO: 117 and the fourth set reverse primer sequence shown in SEQ ID NO: 118;
the fifth set forward primer sequence shown in SEQ ID NO: 121 and the fifth set reverse primer sequence shown in SEQ ID NO: 122;
the sixth set forward primer sequence shown in SEQ ID NO: 125 and the sixth set reverse primer sequence shown in SEQ ID NO: 126;
the seventh set forward primer sequence shown in SEQ ID NO: 129 and the seventh set reverse primer sequence shown in SEQ ID NO: 130; and
the eighth set forward primer sequence shown in SEQ ID NO: 133 and the eighth set reverse primer sequence shown in SEQ ID NO: 134;
wherein the probe:antiprobe system comprises:
the first set iDDS probe sequence shown in SEQ ID NO: 107 and the first set iDDS antiprobe sequence shown in SEQ ID NO: 108;
the second set iDDS probe sequence shown in SEQ ID NO: 111 and the second set iDDS antiprobe sequence shown in SEQ ID NO: 112;
the third set iDDS probe sequence shown in SEQ ID NO: 115 and the third set iDDS antiprobe sequence shown in SEQ ID NO: 116;
the fourth set iDDS probe sequence shown in SEQ ID NO: 119 and the fourth set iDDS antiprobe sequence shown in SEQ ID NO: 120;
the fifth set iDDS probe sequence shown in SEQ ID NO: 123 and the fifth set iDDS antiprobe sequence shown in SEQ ID NO: 124;
the sixth set iDDS probe sequence shown in SEQ ID NO: 127 and the sixth set iDDS antiprobe sequence shown in SEQ ID NO: 128;
the seventh set iDDS probe sequence shown in SEQ ID NO: 131 and the seventh set iDDS antiprobe sequence shown in SEQ ID NO: 132;
the eighth set Mycobacterium chelonae iDDS probe sequence shown in SEQ ID NO: 135 and the eighth set Mycobacterium chelonae iDDS antiprobe sequence shown in SEQ ID NO: 136; and
the eighth set Mycobacterium abscessus iDDS probe sequence shown in SEQ ID NO: 137 and the eighth set Mycobacterium abscessus iDDS antiprobe sequence shown in SEQ ID NO: 138; or combinations thereof; and
wherein the probes are labeled with a fluorophore or a fluorophore and a quencher, and the antiprobes are labeled with a quencher.

37. The multi-probe system of claim 30, wherein one or more probes are configured for sequential or separate use in target detection.

38. The multi-probe system of claim 1, wherein the two or more related signature sequences are in the same gene or species and wherein the probe technology is selected from the group consisting of iDDS probes, iDDS-2Q probes and MacMan probes, said system configured for endpoint detection with simultaneous confirmation thereof.

39. A method for improving signaling and/or reliability in detecting a genus, a species, a gene or other related nucleic acid targets, said method comprising the steps of:
obtaining a biological sample from a human, an animal or an organism;
contacting the biological sample with the labeled probes and flanking primer components comprising the multi-probe system of claim 1; and
detecting a signal emitted by the label upon contacting the target in the biological sample;
wherein, if the labeled probes comprise an identical label, the signal is amplified, thereby improving signaling in detecting the target, or
wherein, if the labeled probes each comprise a different label, detection is confirmed, thereby improving reliability in detecting the target; or a combination thereof.


SEQUENCE LISTING (138 sequences)

SEQ ID NO: 1 - DNA, 22 bp, Artificial Sequence
  iDDS probe for resistant Enterococcus vancomycin A gene
ctcccgccttttgggttattaa

SEQ ID NO: 2 - DNA, 22 bp, Artificial Sequence
  iDDS antiprobe for resistant Enterococcus vancomycin A gene
ttaataacccaaatggcgggag

SEQ ID NO: 3 - DNA, 21 bp, Artificial Sequence
  iDDS probe for resistant Enterococcus vancomycin A gene
cgcaacgatgtatgtcaacga

SEQ ID NO: 4 - DNA, 21 bp, Artificial Sequence
  iDDS antiprobe for resistant Enterococcus vancomycin A gene
tcgttgacatacctcgttgcg

SEQ ID NO: 5 - DNA, 24 bp, Artificial Sequence
  forward primer for resistant Enterococcus vancomycin A gene
gatattcaaagctcagcaatttgt

SEQ ID NO: 6 - DNA, 17 bp, Artificial Sequence
  reverse primer for resistant Enterococcus vancomycin A gene
tagctgccaccggccta

SEQ ID NO: 7 - DNA, 19 bp, Artificial Sequence
  iDDS probe for resistant Enterococcus vancomycin B gene
atcgccgttcccgaatttc

SEQ ID NO: 8 - DNA, 19 bp, Artificial Sequence
  iDDS antiprobe for resistant Enterococcus vancomycin B gene
gaaattcgggaacggccat

SEQ ID NO: 9 - DNA, 17 bp, Artificial Sequence
  iDDS probe for resistant Enterococcus vancomycin B gene
cgcctccggcttgtcac

SEQ ID NO: 10 - DNA, 17 bp, Artificial Sequence
  iDDS antiprobe for resistant Enterococcus vancomycin B gene
gtgacaagccggtggcg

SEQ ID NO: 11 - DNA, 24 bp, Artificial Sequence
  forward primer for resistant Enterococcus vancomycin B gene
caaatcactggcctacattcttac

SEQ ID NO: 12 - DNA, 22 bp, Artificial Sequence
  reverse primer for resistant Enterococcus vancomycin B gene
cagcgttaagttcttccgtacc

SEQ ID NO: 13 - DNA, 20 bp, Artificial Sequence
  iDDS probe for Trichomonas vaginalis repetitive sequence
cctcggagtctttgaatcgg

SEQ ID NO: 14 - DNA, 20 bp, Artificial Sequence
  iDDS antiprobe for Trichomonas vaginalis repetitive sequence
ccgattcatagactccgagg

SEQ ID NO: 15 - DNA, 24 bp, Artificial Sequence
  iDDS probe for Trichomonas vaginalis repetitive sequence
accaagaatggtgtaactcgacct

SEQ ID NO: 16 - DNA, 24 bp, Artificial Sequence
  iDDS antiprobe for Trichomonas vaginalis repetitive sequence
aggtagagttacaccattcttggt

SEQ ID NO: 17 - DNA, 23 bp, Artificial Sequence
  forward primer for Trichomonas vaginalis repetitive sequence
aatttccgtttaatttcatggtc

SEQ ID NO: 18 - DNA, 21 bp, Artificial Sequence
  reverse primer for Trichomonas vaginalis repetitive sequence
ttygtgtctcgtgccatagtc

SEQ ID NO: 19 - DNA, 19 bp, Artificial Sequence
  iDDS probe for wild type human EGFR gene
agcagtttgggcagcccaa

SEQ ID NO: 20 - DNA, 19 bp, Artificial Sequence
  iDDS antiprobe for wild type human EGFR gene
ttgggctggcctaactgct

SEQ ID NO: 21 - DNA, 17 bp, Artificial Sequence
  iDDS probe for L858R point mutation in human EGFR gene
agcagtttgggccgccc

SEQ ID NO: 22 - DNA, 17 bp, Artificial Sequence
  iDDS antiprobe for L858R point mutation in human EGFR gene
gggcgggccaaacggct

SEQ ID NO: 23 - DNA, 19 bp, Artificial Sequence
  forward primer for human EGFR gene
gaaaacaccgcagcatgtc

SEQ ID NO: 24 - DNA, 23 bp, Artificial Sequence
  reverse primer for human EGFR gene
ctgcatggtattctttctcttcc

SEQ ID NO: 25 - DNA, 22 bp, Artificial Sequence
  iDDS probe for exon 19 deletion in human EGFR gene
ctctggatcccagaaggtgaga

SEQ ID NO: 26 - DNA, 22 bp, Artificial Sequence
  iDDS antiprobe for exon 19 deletion in human EGFR gene
tctctccttctgggatccagag

SEQ ID NO: 27 - DNA, 24 bp, Artificial Sequence
  iDDS probe for exon 19 deletion in human EGFR gene
caaggaattaagagaagcaacatc

SEQ ID NO: 28 - DNA, 24 bp, Artificial Sequence
  iDDS antiprobe for exon 19 deletion in human EGFR gene
gatgttgcctctcttaattccttg

SEQ ID NO: 29 - DNA, 20 bp, Artificial Sequence
  forward primer for exon 19 deletion in human EGFR gene
ggtaacatccacccagatca

SEQ ID NO: 30 - DNA, 20 bp, Artificial Sequence
  reverse primer for exon 19 deletion in human EGFR gene
catcgaggatttccttgttg

SEQ ID NO: 31 - DNA, 21 bp, Artificial Sequence
  iDDS probe for an untranslated region of Echovirus DNA
cggaaccgactactttgggtg

SEQ ID NO: 32 - DNA, 21 bp, Artificial Sequence
  iDDS antiprobe for an untranslated region of Echovirus DNA
cacccaatgtagtcggttccg

SEQ ID NO: 33 - DNA, 24 bp, Artificial Sequence
  iDDS probe for an untranslated region of Echovirus DNA
aaacacggacacccaaagtagtcg

SEQ ID NO: 34 - DNA, 24 bp, Artificial Sequence
  iDDS antiprobe for an untranslated region of Echovirus DNA
cgactactttggttgtccgtgttt

SEQ ID NO: 35 - DNA, 18 bp, Artificial Sequence
  forward primer for an untranslated region of Echovirus DNA
cccctgaatgcggmtaat

SEQ ID NO: 36 - DNA, 21 bp, Artificial Sequence
  reverse primer for an untranslated region of Echovirus DNA
rattgtcaccataagcagcca

SEQ ID NO: 37 - DNA, 22 bp, Artificial Sequence
  iDDS probe for an untranslated region of Parechovirus DNA
cactatggatctgatctggggc

SEQ ID NO: 38 - DNA, 22 bp, Artificial Sequence
  iDDS antiprobe for an untranslated region of Parechovirus DNA
gccccagatcagatccttagtg

SEQ ID NO: 39 - DNA, 19 bp, Artificial Sequence
  iDDS probe for an untranslated region of Parechovirus DNA
cgggtaccttctgggcatc

SEQ ID NO: 40 - DNA, 19 bp, Artificial Sequence
  iDDS antiprobe for an untranslated region of Parechovirus DNA
gatgccctgaaggtacccg

SEQ ID NO: 41 - DNA, 19 bp, Artificial Sequence
  forward primer for an untranslated region of Parechovirus DNA
ggccaaaagccaaggttta

SEQ ID NO: 42 - DNA, 20 bp, Artificial Sequence
  reverse primer for an untranslated region of Parechovirus DNA
ttggcccactagacgttttt

SEQ ID NO: 43 - DNA, 18 bp, Artificial Sequence
  IDDS probe for Human Papillomavirus 16 L1 gene
cgagcacagggccacaat

SEQ ID NO: 44 - DNA, 18 bp, Artificial Sequence
  IDDS antiprobe for Human Papillomavirus 16 L1 gene
attgtggtcctgtgctcg

SEQ ID NO: 45 - DNA, 20 bp, Artificial Sequence
  IDDS probe for Human Papillomavirus 16 L1 gene
ggttaccccaacaaatgcca

SEQ ID NO: 46 - DNA, 20 bp, Artificial Sequence
  IDDS antiprobe for Human Papillomavirus 16 L1 gene
tggcttttgttggggtaacc

SEQ ID NO: 47 - DNA, 22 bp, Artificial Sequence
  forward primer for Human Papillomavirus 16 L1 gene
ggttacctctgatgcccaaata

SEQ ID NO: 48 - DNA, 24 bp, Artificial Sequence
  reverse primer for Human Papillomavirus 16 L1 gene
tttctgaagtagatatggcagcac

SEQ ID NO: 49 - DNA, 26 bp, Artificial Sequence
  IDDS probe for Human Papillomavirus 18 L1 gene
ggttacataaggcacagggtcataac

SEQ ID NO: 50 - DNA, 26 bp, Artificial Sequence
  IDDS antiprobe for Human Papillomavirus 18 L1 gene
gttatgaccctgtcccttatgtaacc

SEQ ID NO: 51 - DNA, 22 bp, Artificial Sequence
  IDDS probe for Human Papillomavirus 18 L1 gene
ttgattatgccagcaaacacca

SEQ ID NO: 52 - DNA, 22 bp, Artificial Sequence
  IDDS antiprobe for Human Papillomavirus 18 L1 gene
tggagtttgctggcataatcaa

SEQ ID NO: 53 - DNA, 22 bp, Artificial Sequence
  forward primer for Human Papillomavirus 18 L1 gene
tgttacctctgactcccagttg

SEQ ID NO: 54 - DNA, 20 bp, Artificial Sequence
  reverse primer for Human Papillomavirus 18 L1 gene
ttgcccaggtacaggagact

SEQ ID NO: 55 - DNA, 20 bp, Artificial Sequence
  IDDS probe for seasonal H1N1 influenza A hemagglutinin segment
ttgggtaactgcagcgttgc

SEQ ID NO: 56 - DNA, 20 bp, Artificial Sequence
  IDDS antiprobe for seasonal H1N1 influenza A hemagglutinin segment
gcaacgcagcagttacccaa

SEQ ID NO: 57 - DNA, 22 bp, Artificial Sequence
  forward primer for seasonal H1N1 influenza A hemagglutinin segment
gctaccatgctaacaactcgac

SEQ ID NO: 58 - DNA, 17 bp, Artificial Sequence
  reverse primer for seasonal H1N1 influenza A hemagglutinin segment
tcgcattctgggtttcc

SEQ ID NO: 59 - DNA, 23 bp, Artificial Sequence
  iDDS probe for 2009-pandemic H1N1 influenza A hemagglutinin segment
tccgatcacaattggaaaatgtc

SEQ ID NO: 60 - DNA, 23 bp, Artificial Sequence
  iDDS antiprobe for 2009-pandemic H1N1 influenza A hemagglutinin segment
gactttttccaattgtgatcgga

SEQ ID NO: 61 - DNA, 20 bp, Artificial Sequence
  forward primer for 2009-pandemic H1N1 influenza A hemagglutinin segment
ataaacaccagcctcccatt

SEQ ID NO: 62 - DNA, 20 bp, Artificial Sequence
  reverse primer for 2009-pandemic H1N1 influenza A hemagglutinin segment
gtggccagtctcaattttgt

SEQ ID NO: 63 - DNA, 22 bp, Artificial Sequence
  iDDS probe for H3N2 influenza A hemagglutinin segment
tatgatgtgccggattatgcct

SEQ ID NO: 64 - DNA, 22 bp, Artificial Sequence
  iDDS antiprobe for H3N2 influenza A hemagglutinin segment
aggcataatccggctcatcata

SEQ ID NO: 65 - DNA, 20 bp, Artificial Sequence
  forward primer for H3N2 influenza A hemagglutinin segment
cctttttgttgaacgaagca

SEQ ID NO: 66 - DNA, 22 bp, Artificial Sequence
  reverse primer for H3N2 influenza A hemagglutinin segment
tcattgttaaactccagtgtgc

SEQ ID NO: 67 - DNA, 23 bp, Artificial Sequence
  iDDS probe for influenza B hemagglutinin segment
ccgtgaccagtctaattgtctcc

SEQ ID NO: 68 - DNA, 23 bp, Artificial Sequence
  iDDS antiprobe for influenza B hemagglutinin segment
ggagacatttagactggtcacgg

SEQ ID NO: 69 - DNA, 21 bp, Artificial Sequence
  forward primer for influenza B hemagglutinin segment
gtcaagagcaccgattatcac

SEQ ID NO: 70 - DNA, 23 bp, Artificial Sequence
  reverse primer for influenza B hemagglutinin segment
caaccatgtcagctattatggag

SEQ ID NO: 71 - DNA, 22 bp, Artificial Sequence
  iDDS probe for human T-cell receptor excision circles gene
ttgtaaaggtgcccactcctgt

SEQ ID NO: 72 - DNA, 22 bp, Artificial Sequence
  iDDS antiprobe for human T-cell receptor excision circles gene
acaggagtgggctcctttacaa

SEQ ID NO: 73 - DNA, 20 bp, Artificial Sequence
  iDDS probe for human T-cell receptor excision circles gene
tgcaggtgcctatgcatcac

SEQ ID NO: 74 - DNA, 20 bp, Artificial Sequence
  iDDS antiprobe for human T-cell receptor excision circles gene
gtgatgcttaggcacctgca

SEQ ID NO: 75 - DNA, 20 bp, Artificial Sequence
  forward primer for human T-cell receptor excision circles gene
ttcaaccatgctgacacctc

SEQ ID NO: 76 - DNA, 18 bp, Artificial Sequence
  reverse primer for human T-cell receptor excision circles gene
cagctgcagggtttaggc

SEQ ID NO: 77 - DNA, 40 bp, Artificial Sequence
  Half-Universal probe for human immunodeficiency virus Gag gene
ctacgtagactagacgttcacctatcccagtgggagagat

SEQ ID NO: 78 - DNA, 19 bp, Artificial Sequence
  Half-Universal antiprobe for human immunodeficiency virus Gag gene
gaacgtctagtctacgtag

SEQ ID NO: 79 - DNA, 24 bp, Artificial Sequence
  primer for human immunodeficiency virus Gag gene
atgctgacagggctatacattctt

SEQ ID NO: 80 - DNA, 37 bp, Artificial Sequence
  Half-Universal probe for human immunodeficiency virus Pol gene
ctacgtagactagacgttccagagcagaccagagcca

SEQ ID NO: 81 - DNA, 23 bp, Artificial Sequence
  primer for human immunodeficiency virus Pol gene
aggggtcgttgccaaagagtgat

SEQ ID NO: 82 - DNA, 24 bp, Artificial Sequence
  iDDS probe for human isocitrate dehydrogenase 1 gene
cataggtcatgatgcttatgggga

SEQ ID NO: 83 - DNA, 24 bp, Artificial Sequence
  iDDS antiprobe for human isocitrate dehydrogenase 1 gene point mutation
tccgcataagcatgatgacctatg

SEQ ID NO: 84 - DNA, 24 bp, Artificial Sequence
  iDDS probe for human isocitrate dehydrogenase 1 gene point mutation
ccataagcatgacaagctatgatg

SEQ ID NO: 85 - DNA, 24 bp, Artificial Sequence
  iDDS antiprobe for human isocitrate dehydrogenase 1 gene point mutation
catcataggttgtcttgcttatgg

SEQ ID NO: 86 - DNA, 20 bp, Artificial Sequence
  forward primer for human isocitrate dehydrogenase 1 gene
ggcttgtgagtggatgggta

SEQ ID NO: 87 - DNA, 22 bp, Artificial Sequence
  reverse primer for human isocitrate dehydrogenase 1 gene
aaaacatgcaaaatcacattat

SEQ ID NO: 88 - DNA, 18 bp, Artificial Sequence
  iDDS probe for human isocitrate dehydrogenase 2 gene point mutation
ccattggcaaggacgccc

SEQ ID NO: 89 - DNA, 18 bp, Artificial Sequence
  iDDS antiprobe for human isocitrate dehydrogenase 2 gene point mutation
gggcgtgcttgcctatgg

SEQ ID NO: 90 - DNA, 18 bp, Artificial Sequence
  iDDS probe for human isocitrate dehydrogenase 2 gene point mutation
ccattggcatggacgccc

SEQ ID NO: 91 - DNA, 18 bp, Artificial Sequence
  iDDS antiprobe for human isocitrate dehydrogenase 2 gene point mutation
gggcgtgcatgcctatgg

SEQ ID NO: 92 - DNA, 20 bp, Artificial Sequence
  forward primer for human isocitrate dehydrogenase 2 gene
cactattatctctgtcctca

SEQ ID NO: 93 - DNA, 17 bp, Artificial Sequence
  reverse primer for human isocitrate dehydrogenase 2 gene
agacaagaggatggcta

SEQ ID NO: 94 - DNA, 25 bp, Artificial Sequence
  forward primer for adjacent reference-mutant regions in exon 2, codon 12 or 13 of human KRAS gene
cctgctgaaaatgactgaatataaa

SEQ ID NO: 95 - DNA, 16 bp, Artificial Sequence
  iDDS probe for adjacent reference-mutant regions in exon 2, codon 12 or 13 of human KRAS gene
cctacgccaccagctc

SEQ ID NO: 96 - DNA, 16 bp, Artificial Sequence
  iDDS antiprobe for adjacent reference-mutant regions in exon 2, codon 12 or 13 of human KRAS gene
gaggtggtggcgtagg

SEQ ID NO: 97 - DNA, 22 bp, Artificial Sequence
  ZIPR probe for adjacent reference-mutant regions in exon 2, codon 12 or 13 of human KRAS gene
tggatcatattcgtccacaaaa

SEQ ID NO: 98 - DNA, 22 bp, Artificial Sequence
  ZIPR antiprobe for adjacent reference-mutant regions in exon 2, codon 12 or 13 of human KRAS gene
ttttgaggacgaatatgatcca

SEQ ID NO: 99 - DNA, 26 bp, Artificial Sequence
  flanking primer for adjacent wild type-mutant regions in codon 600 of human BRAF gene
tgaagacctcacagtaaaaataggtg

SEQ ID NO: 100 - DNA, 20 bp, Artificial Sequence
  flanking primer for adjacent wild type-mutant regions in codon 600 of human BRAF gene
ccacaaaatggatccagaca

SEQ ID NO: 101 - DNA, 22 bp, Artificial Sequence
  iDDS probe for mutant region in codon 600 of human BRAF gene
agctactgagaaatctcgatgg

SEQ ID NO: 102 - DNA, 22 bp, Artificial Sequence
  iDDS antiprobe for mutant region in codon 600 of human BRAF gene
ccatcgagatttctgtgtagct

SEQ ID NO: 103 - DNA, 21 bp, Artificial Sequence
  iDDS probe for wild type region in codon 600 of human BRAF gene
caactcatcacggagctcatg

SEQ ID NO: 104 - DNA, 21 bp, Artificial Sequence
  iDDS antiprobe for wild type region in codon 600 of human BRAF gene
catgagctgcgtgatgtgttg

SEQ ID NO: 105 - DNA, 17 bp, Artificial Sequence
  forward primer for Pseudomonas aeruginosa algD gene
cggggctcgtagtaacg

SEQ ID NO: 106 - DNA, 19 bp, Artificial Sequence
  reverse primer for Pseudomonas aeruginosa algD gene
cgacatagcccaaaccaaa

SEQ ID NO: 107 - DNA, 20 bp, Artificial Sequence
  iDDS probe for Pseudomonas aeruginosa algD gene
tgaatgcgatgcgaatcagc

SEQ ID NO: 108 - DNA, 20 bp, Artificial Sequence
  iDDS antiprobe for Pseudomonas aeruginosa algD gene
gctgattcgcttcgcattca

SEQ ID NO: 109 - DNA, 20 bp, Artificial Sequence
  forward primer for Burkholderia cepacia complex recA gene
ggctcgatcaagaagaacga

SEQ ID NO: 110 - DNA, 18 bp, Artificial Sequence
  reverse primer for Burkholderia cepacia complex recA gene
gacgatcttcgcctgcac

SEQ ID NO: 111 - DNA, 18 bp, Artificial Sequence
  iDDS probe for Burkholderia cepacia complex recA gene
cgttccgcgaagcgatct

SEQ ID NO: 112 - DNA, 18 bp, Artificial Sequence
  iDDS antiprobe for Burkholderia cepacia complex recA gene
agattgcttcgcggaacg

SEQ ID NO: 113 - DNA, 21 bp, Artificial Sequence
  forward primer for Stenotrophomonas maltophilia 23S gene
cagagygttttcctagaacca

SEQ ID NO: 114 - DNA, 25 bp, Artificial Sequence
  reverse primer for Stenotrophomonas maltophilia 23S gene
gtatcctgcagtgaatacatagctg

SEQ ID NO: 115 - DNA, 19 bp, Artificial Sequence
  iDDS probe for Stenotrophomonas maltophilia 23S gene
accaggttcgcttccagca

SEQ ID NO: 116 - DNA, 19 bp, Artificial Sequence
  iDDS antiprobe for Stenotrophomonas maltophilia 23S gene
tgctggtagcgaacctggt

SEQ ID NO: 117 - DNA, 19 bp, Artificial Sequence
  forward primer for Achromobacter species OXA-114-like gene
gagccggtctggaactacc

SEQ ID NO: 118 - DNA, 18 bp, Artificial Sequence
  reverse primer for Achromobacter species OXA-114-like gene
tggtgtagcgctggaagc

SEQ ID NO: 119 - DNA, 22 bp, Artificial Sequence
  iDDS probe for Achromobacter species OXA-114-like gene
cgctggatcaagtattcggtgg

SEQ ID NO: 120 - DNA, 22 bp, Artificial Sequence
  iDDS antiprobe for Achromobacter species OXA-114-like gene
cctccgaatacttgatccagcg

SEQ ID NO: 121 - DNA, 23 bp, Artificial Sequence
  forward primer for Staphylococcus aureus femA gene
acagatagcatgccatacagtca

SEQ ID NO: 122 - DNA, 22 bp, Artificial Sequence
  reverse primer for Staphylococcus aureus femA gene
gcagtaagtaagcaagctgcaa

SEQ ID NO: 123 - DNA, 20 bp, Artificial Sequence
  iDDS probe for Staphylococcus aureus femA gene
cacgcaaactgttggccact

SEQ ID NO: 124 - DNA, 20 bp, Artificial Sequence
  iDDS antiprobe for Staphylococcus aureus femA gene
agtggctaacagtttgcgtg

SEQ ID NO: 125 - DNA, 22 bp, Artificial Sequence
  forward primer for Staphylococcus aureus mecA gene
ctacggtaacattgatcgcaac

SEQ ID NO: 126 - DNA, 26 bp, Artificial Sequence
  reverse primer for Staphylococcus aureus mecA gene
tttcaatatgtatgctttggtctttc

SEQ ID NO: 127 - DNA, 22 bp, Artificial Sequence
  iDDS probe for Staphylococcus aureus mecA gene
tgcattcctggaataatgacgc

SEQ ID NO: 128 - DNA, 22 bp, Artificial Sequence
  iDDS antiprobe for Staphylococcus aureus mecA gene
gcgtccttattccaggaatgca

SEQ ID NO: 129 - DNA, 18 bp, Artificial Sequence
  forward primer for Mycobacterium avium complex intergenic spacer gene
ggagcaccacgaaaagca

SEQ ID NO: 130 - DNA, 18 bp, Artificial Sequence
  reverse primer for Mycobacterium avium complex intergenic spacer gene
atggagggactccacacg

SEQ ID NO: 131 - DNA, 22 bp, Artificial Sequence
  iDDS probe for Mycobacterium avium complex intergenic spacer gene
caacagcaaatgattgccagac

SEQ ID NO: 132 - DNA, 22 bp, Artificial Sequence
  iDDS antiprobe for Mycobacterium avium complex intergenic spacer gene
gtctggaaatcatttgctgttg

SEQ ID NO: 133 - DNA, 22 bp, Artificial Sequence
  forward primer for Mycobacterium chelonae and Mycobacterium abscessus intergenic spacer genes
ctttctaaggagcaccatttcc

SEQ ID NO: 134 - DNA, 18 bp, Artificial Sequence
  reverse primer for Mycobacterium chelonae and Mycobacterium abscessus intergenic spacer genes
tatccacggggtggacag

SEQ ID NO: 135 - DNA, 24 bp, Artificial Sequence
  iDDS probe for Mycobacterium chelonae intergenic spacer gene
tctgtagtggttactcgcttggtg

SEQ ID NO: 136 - DNA, 24 bp, Artificial Sequence
  iDDS antiprobe for Mycobacterium chelonae intergenic spacer gene
cttcaagcgagtaaccactacaga

SEQ ID NO: 137 - DNA, 28 bp, Artificial Sequence
  iDDS probe for Mycobacterium abscessus intergenic spacer gene
gaatgaactagggaacataaagtaggca

SEQ ID NO: 138 - DNA, 28 bp, Artificial Sequence
  iDDS antiprobe for Mycobacterium abscessus intergenic spacer gene
tgcctacttttagttccctagttcattc